SEQUENCE LISTING (4 sequences)

SEQ ID NO: 1 - DNA, 399 bp, Rattus norvegicus
  CDS (1)...(399): wild-type rat intestinal fatty acid binding protein cDNA sequence
atggcatttgatggcacttggaaagtataccggaatgagaactatgaaaagttcatggagaaaatgggcattaacgtggtgaagaggaagcttggagctcatgacaacttgaaactgacgatcacacaggaaggaaataaattcacagtcaaagaatcaagcaacttccgaaacattgatgttgtgtttgaactcggcgtcgactttgcctatagtctagcagatggaacagaactcactgggaccttgaccatggagggaaataaacttgttggaaaattcaaacgtgtagacaatggaaaggagctgattgctgtccgagagatttctggtaacgaactaatccaaacctacacatatgaaggagtggaggccaagcgcatctttaagaaggaatag

SEQ ID NO: 2 - protein, 131 aa, Rattus norvegicus
  PEPTIDE (1)...(131): wild-type rat intestinal fatty acid binding protein
AFDGTWKVDRNENYEKFMEKMGINVVKRKLGAHDNLKLTITQEGNKFTVKESSNFRNIDVVFELGVDFAYSLADGTELTGTWTMEGNKLVGKFKRVDNGKELIAVREISGNELIQTYTYEGVEAKRIFKKE

SEQ ID NO: 3 - DNA, 426 bp, Rattus norvegicus
  mutation (1)...(426): rat intestinal fatty acid binding protein DNA sequence coding for substitution of alanine for leucine at position 72
atggcatttgatggcacttggaaagtagaccggaatgagaactatgaaaagttcatggagaaaatgggcattaacgtggtgaagaggaagcttggagctcatgacaacttgaaactgacgatcacacaggaaggaaataaattcacagtcaaagaatcaagcaacttccgaaacattgatgttgtgtttgaactcggcgtcgactttgcctatagtgctgcagatggaacagaactcaccggtacctggacaatggagggaaataaacttgttggaaagtttaaacgtgtagacaatggaaaggagctgattgctgtccgagagatttctggtaacgaactaatccagacctacacatatgaaggagtggaggccaagcggatctttaagaaggaccgcggtcatcatcaccatcatcactagtaa

SEQ ID NO: 4 - protein, 139 aa, Rattus norvegicus
  MUTAGEN (72)...(72): rat intestinal fatty acid binding protein with alanine substitution for leucine at position 72
AFDGTWKVDRNENYEKFMEKMGINVVKRKLGAHDNLKLTITQEGNKFTVKESSNFRNIDVVFELGVDFAYSAADGTELTGTWTMEGNKLVGKFKRVDNGKELIAVREISGNELIQTYTYEGVEAKRIFKKDRGHHHHHH

(19) United States
(12) Patent Application Publication
(10) Pub. No.: US 2006/0257938 A1
(43) Pub. Date: Nov. 16, 2006

(54) Method of screening for drugs that block ligand binding to a lipid binding protein

(76) Inventors: Alan Marc Kleinfeld, La Jolla, CA (US); James Patrick Kampf, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/430,083

(22) Filed: May 8, 2006

Related U.S. Application Data
(60) Provisional application No. 60/679,921, filed on May 10, 2005.

(51) Int. Cl. G01N 33/53

(52) U.S. Cl. 435/7.1

(57) ABSTRACT

Methods are disclosed to screen for drugs that interfere with the binding between a specific lipid-binding protein and a selected ligand. Successful candidates have potential in disease treatments, particularly diabetes and some forms of cancer.

### RELATED APPLICATIONS

This application claims priority to provisional application 60/679,921 filed May 10, 2005 which is incorporated herein by reference.

### BACKGROUND OF THE INVENTION

1. Field of the Invention

Embodiments are directed to methods of screening for small molecules with potential as a drug treatment which alter or affect the binding of intracellular lipid binding proteins to a ligand. For example, drugs affecting the binding of a fatty acid to a specific fatty acid binding protein may be identified.

2. Description of the Related Art

Intracellular lipid binding proteins (LiBPs) are small, about 13-15 kDa, water soluble proteins with four recognized subfamilies. Subfamily I contains proteins specific for vitamin A derivatives such as retinoic acid and retinol. Subfamily II contains proteins with specificities for bile acids, eiconsanoids, and heme. Subfamily III contains intestinal type fatty acid binding proteins (FABPs) and Subfamily IV contains all other types of fatty acid binding protein (Haunerland, et al. (2004) Progress in Lipid Research vol. 43: 328-349).

The LiBPs of Subfamilies III and IV, the fatty acid binding proteins (FABPs), are ubiquitous and abundant intracellular proteins. These proteins are expressed at high levels in a number of tissue types [Glatz, et al. Prostaglandins Leukot. Essent. Fatty Acids 48, 33-41 (1993)] and universally among vertebrates [Schaap, et al. Mol. Cell Biochem. 239, 69-77 (2002)]. Although the different FABPs have distinct amino acid sequences and diverse affinities for the different free fatty acids (FFA), all have a molecular mass around 15 kDa and share a common tertiary structure. Crystal structures reveal that LiBPs and therefore the FABPs have a β barrel or “clam shell” structure formed by two orthogonal β-sheets, the interior of which forms the binding pocket for free fatty acids [Sacchettini, J. C., et al. J. Mol. Biol. 208, 327-339 (1989)]. Two α-helices cap the opening to the β barrel. Most of the FABPs bind one FFA molecule within the pocket, with the exception of liver FABP which can accommodate two FFA molecules per protein [Thompson, J., et al. J. Biol. Chem. 272, 7140-7150 (1997)]. Although the three dimensional structures of the polypeptide backbones of these proteins are virtually identical, the distinct expression patterns and diverse affinities for their ligands imply that the different proteins may have unique metabolic functions.

Although the exact function of these different FABPs is not entirely clear, it is likely that they are involved in various aspects of normal FFA trafficking and metabolism [Weisiger, R. A., et al. Am. J. Physiol Gastrointest. Liver Physiol 282, G105-G115 (2002); Hsu, K. T., et al. J. Biol. Chem. 271, 13317-13323 (1996); Shen, W. J., et al. Proc. Natl. Acad. Sci. U.S.A 96, 5528-5532 (1999); Binas, B., et al. FASEB J. 13, 805-812 (1999)]. Moreover knock-out studies, for example of the adipocyte FABP (A-FABP) in adipocytes, raise the possibility that A-FABP may promote insulin resistance while A-FABP knock-outs in macrophages suggest that A-FABP plays an important role in promoting atherogenesis [Makowski, L. et al., Nat. Med. 7, 699-705 (2001); Boord, J. B., et al. Circulation 110, 1492-1498 (2004)]. Other studies suggest that fatty acid metabolism may play an important role in tumorigenesis [Richieri, G. V., et al. J. Immunol. 147, 2809-2815 (1991); Kleinfeld A. M., et al. Free fatty acid release from human breast cancer tissue inhibits CTL-mediated killing. Submitted (2005)]. Cancer cells release high levels of FFA and elevated levels of FFA inhibit killing by cytotoxic T lymphocytes (CTL) [Richieri,G. V., et al. J. Immunol. 145, 1074-1077 (1990)]. These studies suggest that FFA release from tumor cells may prevent CTL-mediated clearance of the tumor and that drugs capable of inhibiting this release could significantly augment anti-cancer immune therapy. Fatty acid binding proteins have been linked to the development of cancerous tumors, presumably through their role in fatty acid metabolism, but their function is not well known and their effects seem somewhat contradictory [Hashimoto, et al. Pathobiology 71, 267-273 (2004); Adamson,J. et al. Oncogene 22, 2739-2749 (2003), respectively]. In the former case, heart FABP has a positive association with levels of fatty acid synthase, a possible source of the observed increase in FFA [Kuhajda, F. P., et al. Proc. Natl. Acad. Sci. U.S.A 97, 3450-3454 (2000)]. On the other hand, over-expression of adipocyte-FABP has been shown to induce apoptosis in prostate cancer cells [De Santis, M. L., et al. J. Exp. Ther. Oncol. 4, 91-100 (2004)].

The ability to inhibit natural ligand binding to LiBPs with non-native ligand molecules would provide a method to characterize the fimctions of the LiBPs and to modify specific lipid trafficking and metabolic fuictions of these proteins. This method would provide a way to knock-out the intracellular FFA binding of FABPs and the intracellular lipid binding proteins of their ligands. Such specific inhibitors would likely prove usefuil as therapeutic agents for treatment of diseases, especially metabolic diseases. If the adipocyte FABP could be specifically inhibited, for example, it may offer a means to treat type II diabetes or atherosclerosis as implied by the before-mentioned studies relating this FABP to insulin resistance and atherogenesis.

Searches for FABP inhibitors using displacement of the fluorophore ANS [U.S. Pat. No. 6,548,529; 6,649,622; 6,670,380; 6,919,323; 6,927,227; 6,984,645] or fluorescence polarization [Lehman et al. Bioorg. Med. Chem. Lett. 14, 4445-4448 (2004), Ringom et al Bioorg. Med. Chem. Lett. 14, 4449-4452 (2004)] reveal large numbers of non-FFA componds capable of displacing a fluorophore from an FABP. Unfortunately the highest binding affinities of these compounds were in the 1 μM range and are therefore unsuitable as inhibitors/drugs for FABPs which have binding affinities for the physiologically most abundant FFA that range from about 1 to 80 nM. The absence of high affinity inhibitors/drugs found among the large libraries of compounds in these studies likely reflects the low sensitivity of the assay methods used in these studies. In contrast the methods described here are capable of detecting compounds with sub-nanomolar affinities.

### SUMMARY OF THE INVENTION

Embodiments of the invention are directed to various screening methods to identify agents with high affinity for LiBP. These screening methods may be used separately or in combination.

One embodiment is directed to a method of identifying agents with high affinity for a fluorescently labeled LiBP (a LiBP probe) which may include the followings steps. A first fluorescence is measured for a fluorescently labeled LiBP probe. The LiBP probe is incubated with the agent. In some embodiments, the agent has been preselected by one of the described methods. A second fluorescence is measured. The first fluorescence of the LiBP probe in the absence of the agent is compared to a second fluorescence in the presence of the agent. Agents are selected that affect a difference between the first fluorescence and the second fluorescence. A difference between the first fluorescence and the second fluorescence indicates that the agent has an affinity for the LiBP probe.

In preferred embodiments, the LiBP probe is a variant of the amino acid sequence shown as SEQ ID NO: 2, preferably a variant having one or more substitutions, insertions and/or deletions in the amino acid sequence shown as SEQ ID NO: 2. More preferably, the LiBP probe is ADIFAB or ADIFAB2 or any of the probes described in Tables 1-6. Preferably, the agent is a drug candidate.

Embodiments of the invention are directed to a method of screening for an agent that modulates the binding function of a LiBP which may include the followings steps. A wild type LiBP is reacted with a fluorescence indicator, where the fluorescence indicator is non-covalently bound in a binding pocket of the wild type LiBP to form a LiBP binding complex. The agent to be tested is contacted with the LiBP complex. In some embodiments, the agent has been preselected by one of the described methods. Agents are identified that displace the fluorescence indicator, thereby changing fluorescence.

In some embodiments of the invention, the wild type LiBP is titrated with the fluorescent indicator to determine the binding constant of the fluorescent indicator with the LiBP. The wild type LIBP is titrated with an agent, which may be an agent which has been selected by one of the methods above, to determine a binding constant for each selected agent by using a standard competition assay and the binding constant of the fluorescent indicator. The binding constants are evaluated to identify agents that modulate the binding function of the LiBP.

In preferred embodiments, the fluorescence indicator includes a fatty acid labeled with a fluorescent indicator. In preferred embodiments, the wild type LiBP is a fatty acid binding protein.

Embodiments of the invention are directed to a method of screening for an agent which may include the following steps. A composition which includes a wild type LiBP and a probe is added to at least some wells of a multi-well plate. Test agents are added to the wells. These test agents may or may not have been preselected by a method as described herein. Fluorescence of each well is measured to determine the degree of binding of each agent to the wild type LiBP. Agents are selected that bind to the wild type LiBP. The wild type LiBP and the probe are titrated with the selected agents to determine binding constants, and high affinity agents are identified.

Preferably, the probe includes a LiBP, covalently labeled with a fluorescent molecule. In some preferred embodiments, the LiBP of the probe is the same as the wild type LiBP. In preferred embodiments, the LiBP is a fatty acid binding protein.

In preferred embodiments, the LiBP probe is a variant of the amino acid sequence shown as SEQ ID NO: 2, preferably a variant having one or more substitutions, insertions and/or deletions in the amino acid sequence shown as SEQ ID NO: 2. More preferably, the LiBP probe is ADIFAB or ADIFAB2 or any of the probes described in Tables 1-6. Preferably, the agent is a drug candidate.

Embodiments of the invention are directed to a method of selecting for high affinity agents which are permeant to cells of interest which may include the following steps. A probe is transfected into a cell. Any selected agent, including any of the agents selected by the methods as described above, is tested for ability to enter the cell by monitoring the change in probe fluorescence after adding the agent to the outside of the cell. High affinity agents which are permeant to cells of interest are then selected.

Preferably, the cell is a mammalian cell. In preferred embodiments, transfection is by microinjection, electroporation, use of lipid or peptide transfection reagents, or mechanical membrane disruption as in scrape, scratch, bead, or syringe loading.

In preferred embodiments, the probe is a variant of the amino acid sequence shown as SEQ ID NO: 2, preferably a variant having one or more substitutions, insertions and/or deletions in the amino acid sequence shown as SEQ ID NO: 2. More preferably, the LiBP probe is ADIFAB or ADIFAB2 or any of the probes described in Tables 1-6. Preferably, the agent is a drug candidate.

Further aspects, features and advantages of this invention will become apparent from the detailed description of the preferred embodiments which follow.

### BRIEF DESCRIPTION OF THE DRAWINGS

These and other feature of this invention will now be described with reference to a drawing which is intended to illustrate and not to limit the invention.

The FIGURE shows a time course as hit compound (A9) crosses the plasma membrane and binds intracellular ADIFAB.

### DETAILED DESCRIPTION OF THE PREFERRED EMBODIMENT

While the described embodiment represents the preferred embodiment of the present invention, it is to be understood that those skilled in the art can modify the process without departing from the spirit of the invention. Preferred embodiments of the present invention relate to screening for agents that effect the binding of a selected lipid binding protein to its natural hydrophobic metabolite. More particularly, the invention relates to the use of such agents for clinical medicine, drug development and basic science.

Embodiments of the invention provide efficient methods of identifying agents, compounds or lead compounds for agents active at the level of a LiBP alterable cellular function. Preferred embodiments utilize a high throughput screening method to screen chemical libraries for lead compounds. In preferred embodiments, these compounds are then subjected to further screening to identify compounds which are capable of modulation of the activity of a LiBP in vivo. Identified reagents find use in the pharmaceutical industries for animal and human trials; for example, the reagents may be derivatized and rescreened in in vitro and in vivo assays to optimize activity and minimize toxicity for pharmaceutical development. Target indications include cardiac disease, stroke, neurological diseases such as dementia and Alzheimer's disease, diabetes, inflammatory diseases, obesity, metabolic syndrome and certain cancers etc. The ability of an agent to bind to an LiBP protein and further to displace an LiBP ligand indicates that the agent is capable of modulating the binding characteristics of the LiBP protein.

Probes are LiBPs that have been ‘labeled’ through the covalent addition of a fluorescent molecule (fluorophore) to a specific site on the protein and that bind metabolites in vivo. Probes have the characteristic that their fluorescence changes in a measurable way when they bind metabolites. The ability of an agent to bind to the probe can then be assessed by measuring the change in fluorescence upon addition of defined concentrations of the agent.

In a preferred embodiment, the probe is a fatty acid binding protein (FABP), more preferably a recombinant rat intestinal fatty acid binding protein (rI-FABP), which has been derivatized with acrylodan. DNA and protein sequences for Fatty Acid Binding Proteins (FABPs) are shown in the sequence listing. SEQ ID NO: 1 shows the cDNA sequence for the wild-type rat intestinal Fatty Acid Binding Protein (rIFABP). The rat fatty acid binding protein is post-translationally modified in the rat, with the modifications including the removal of the N-terminal methionine and the acetylation of the “new” N-terminal residue Ala. Protein sequences are numbered starting with the first residue of the mature protein. Thus, Ala is residue 1 in the corresponding protein shown as SEQ ID NO: 2.

Derivatization is performed using known methods substantially as previously described (U.S. Pat. No. 5,470,714 & Richieri, G. V, et al., J. Biol. Chem., (1992) 276: 23495-23501), and the resulting probe (ADIFAB) is commercially available (FFA Sciences LLC, San Diego, Calif.). A different fluorescence is exhibited by ADIFAB when FFA is bound and the concentration of unbound FFA (FFAu) can be determined from the change in fluorescence. The wavelength emitted by the fluorescently-labeled FABP depends upon the label and protein used.

In an alternate preferred embodiment, the protein is rI-FABP that has Ala substituted for Leu at position 72 (rI-FABP-L72A) with the resulting probe named ADIFAB2. SEQ ID NO: 3 shows the DNA sequence for rI-FABP-L72A, which is the DNA sequence encoding the protein for ADIFAB2 probe. SEQ ID NO: 4 shows the ADIFAB2 probe protein sequence. Other probes useful in embodiments of the invention are shown in Tables 1-6 and are also described in U.S. application Ser. No. 11/085,792, filed Mar. 21, 2005 which is incorporated herein by reference. The indicated substitutions are with reference to the ADIFAB protein of SEQ ID NO: 2.

TABLE 1
List of responsive clones excluding L72A and
WT IFABP
Substitutions are shown with respect to SEQ ID NO: 2.

| Clone | AvgOfRo | DR/DR AA | DR/DR LNA | DR/DR LA | DR/DR OA | DR/DR PA |
|---|---|---|---|---|---|---|
| 102A, 72A | 0.24 | 0.45 | 0.44 | 0.35 | 0.52 | 0.20 |
| 102A1, 72A | 0.17 | 0.50 | 0.50 | 0.54 | 0.52 | 0.20 |
| 102C, 72A | 0.24 | 0.14 | 0.12 | 0.12 | 0.11 | 0.09 |
| 102D, 72A | 0.41 | 0.52 | 0.61 | 0.63 | 0.59 | 0.28 |
| 102E, 72A | 0.41 | 0.56 | 0.89 | 0.65 | 0.60 | 0.30 |
| 102F, 72A | 0.25 | 0.95 | 0.53 | 0.73 | 0.64 | 0.43 |
| 102G, 72A | 0.38 | 0.63 | 0.38 | 0.78 | 0.65 | 0.36 |
| 102H, 72A | 0.37 | 0.52 | 0.63 | 0.65 | 0.66 | 0.37 |
| 102H1, 72A | 0.50 | 1.30 | 1.22 | 1.08 | 0.85 | 0.56 |
| 102I, 72A | 0.22 | 1.46 | 1.76 | 1.90 | 1.93 | 1.17 |
| 102K, 72A | 0.39 | 0.47 | 0.51 | 0.55 | 0.48 | 0.23 |
| 102L, 72A | 0.22 | 1.25 | 1.41 | 1.40 | 1.40 | 0.97 |
| 102M1, | 0.16 | 1.12 | 1.15 | 1.17 | 1.21 | 1.01 |
| 72A | | | | | | |
| 102M2, | 0.17 | 0.78 | 0.75 | 0.72 | 0.72 | 0.47 |
| 72A | | | | | | |
| 102M3, | 0.15 | 0.83 | 0.75 | 0.73 | 0.73 | 0.52 |
| 72A | | | | | | |
| 102N, 72A | 0.32 | 0.64 | 0.71 | 0.67 | 0.69 | −1.09 |
| 102P, 72A | 0.34 | 0.50 | 0.56 | 0.54 | 0.49 | 0.39 |
| 102Q, 72A | 0.34 | 0.62 | 0.71 | 0.66 | 0.60 | 0.30 |
| 102R, 72A | 0.33 | 0.51 | 0.57 | 0.60 | 0.56 | 0.25 |
| 102S, 72A | 0.24 | 0.39 | 0.43 | 0.45 | 0.43 | 0.18 |
| 102T, 72A | 0.14 | 0.21 | 0.24 | 0.26 | 0.26 | 0.12 |
| 102V, 72A | 0.18 | 0.46 | 0.51 | 0.67 | 0.57 | 0.35 |
| 102W1, | 0.73 | 0.30 | 0.32 | 0.31 | 0.30 | 0.19 |
| 72A | | | | | | |
| 102W2, | 0.67 | 0.47 | 0.48 | 0.47 | 0.46 | 0.28 |
| 72A | | | | | | |
| 102W3, | 0.70 | 0.44 | 0.45 | 0.45 | 0.40 | 0.25 |
| 72A | | | | | | |
| 102Y, 72A | 0.72 | 0.72 | 0.73 | 0.82 | 0.86 | 0.39 |
| 104D1, 72A | 0.65 | 0.48 | 0.67 | 0.71 | 0.64 | 0.34 |
| 104F1, 72A | 0.35 | 0.66 | 0.42 | 0.47 | 0.45 | 0.27 |
| 104F3, 72A | 0.34 | 0.71 | 0.45 | 0.50 | 0.47 | 0.25 |
| 104G2, | 0.25 | 0.52 | 0.95 | 0.95 | 0.93 | 0.70 |
| 72A | | | | | | |
| 104I1, 72A | 0.16 | 0.28 | 0.30 | 0.30 | 0.31 | 0.23 |
| 104L1, 72A | 0.12 | 0.38 | 0.42 | 0.39 | 0.48 | 0.31 |
| 104M1, | 0.16 | 0.67 | 0.72 | 0.69 | 0.73 | 0.61 |
| 72A | | | | | | |
| 104N1, 72A | 0.40 | 1.14 | 1.06 | 0.67 | 0.71 | 0.52 |
| 104Q1, | 0.43 | 0.24 | 0.15 | 0.13 | 0.14 | 0.14 |
| 72A | | | | | | |
| 104R, 72A | 0.40 | 1.18 | 0.96 | 0.93 | 0.88 | 0.52 |
| 104R1, 72A | 0.47 | 1.14 | 1.13 | 1.22 | 1.20 | 1.00 |
| 104S2, 72A | 0.24 | 1.64 | 1.54 | 1.71 | 1.66 | 1.57 |
| 104T1, 72A | 0.22 | 1.01 | 1.07 | 1.16 | 1.10 | 0.99 |
| 104V1, 72A | 0.16 | 0.63 | 0.73 | 0.73 | 0.76 | 0.63 |
| 104Y1, 72A | 0.88 | 0.36 | 0.28 | 0.26 | 0.24 | 0.14 |
| 106A1, 72A | 0.25 | 0.39 | 0.52 | 0.51 | 0.60 | 0.16 |
| 106C2, 72A | 0.24 | 0.17 | 0.22 | 0.20 | 0.22 | 0.09 |
| 106D3, 72A | 0.30 | 0.66 | 0.77 | 0.80 | 0.64 | 0.26 |
| 106F1, 72A | 0.31 | 0.75 | 0.71 | 0.78 | 0.78 | 0.38 |
| 106G1, | 0.25 | 0.37 | 0.44 | 0.48 | 0.51 | 0.17 |
| 72A | | | | | | |
| 106H2, 72A | 0.31 | 0.75 | 0.73 | 0.78 | 0.82 | 0.33 |
| 106I3, 72A | 0.30 | 0.59 | 0.71 | 0.68 | 0.72 | 0.29 |
| 106L3, 72A | 0.28 | 0.84 | 0.84 | 0.87 | 0.87 | 0.43 |
| 106M1, | 0.26 | 0.68 | 0.62 | 0.62 | 0.65 | 0.28 |
| 72A | | | | | | |
| 106N1, 72A | 0.24 | 0.38 | 0.44 | 0.45 | 0.50 | 0.15 |
| 106Q, 72A | 0.24 | 0.37 | 0.38 | 0.36 | 0.39 | 0.18 |
| 106S2, 72A | 0.26 | 0.32 | 0.40 | 0.36 | 0.48 | 0.15 |
| 106T2, 72A | 0.26 | 0.43 | 0.58 | 0.47 | 0.53 | 0.23 |
| 106V1, 72A | 0.30 | 0.61 | 0.82 | 0.82 | 0.84 | 0.32 |
| 106W, 72A | 0.24 | 1.11 | 1.03 | 1.04 | 0.99 | 0.41 |
| 106Y1, 72A | 0.17 | 0.57 | 0.60 | 0.61 | 0.60 | 0.27 |
| 113M, 72A | 0.18 | 1.33 | 1.38 | 1.46 | 1.42 | 1.19 |
| 115A1, 72A | 0.16 | 1.17 | 1.26 | 1.22 | 1.20 | 1.11 |
| 115D1, 72A | 0.16 | 1.17 | 1.20 | 1.19 | 1.21 | 1.14 |
| 115E1, 72A | 0.22 | 0.12 | 0.10 | 0.15 | 0.10 | 0.08 |
| 115F1, 72A | 0.23 | 1.36 | 1.49 | 1.46 | 1.41 | 0.77 |
| 115G1, | 0.17 | 1.33 | 1.37 | 1.36 | 1.37 | 1.21 |
| 72A | | | | | | |
| 115H1, 72A | 0.15 | 0.89 | 0.70 | 0.74 | 0.68 | 0.51 |
| 115I3, 72A | 0.24 | 0.87 | 0.80 | 0.81 | 0.88 | 0.84 |
| 115K3, 72A | 0.16 | 1.26 | 1.32 | 1.23 | 1.25 | 1.18 |
| 115L3, 72A | 0.17 | 1.26 | 1.29 | 1.24 | 1.25 | 1.15 |
| 115M2, | 0.21 | 1.45 | 1.46 | 1.51 | 1.52 | 1.32 |
| 72A | | | | | | |
| 115N2, 72A | 0.16 | 0.92 | 0.73 | 0.78 | 0.71 | 0.56 |
| 115P1, 72A | 0.27 | −0.17 | 1.35 | 1.37 | 1.26 | 0.70 |
| 115R1, 72A | 0.40 | 1.40 | 1.43 | 1.45 | 1.36 | 0.70 |
| 115S1, 72A | 0.39 | 1.55 | 1.51 | 1.51 | 1.41 | 0.77 |
| 115T3, 72A | 0.21 | 0.57 | 0.54 | 0.45 | 0.46 | 0.49 |
| 115V2, 72A | 0.28 | 0.84 | 0.93 | 0.87 | 0.91 | 0.85 |
| 115W1, | 0.37 | 1.48 | 1.40 | 1.49 | 1.35 | 0.81 |
| 72A | | | | | | |
| 115Y1, 72A | 0.40 | 1.42 | 1.27 | 1.38 | 1.23 | 0.73 |
| 117A, 72A | 0.28 | 1.27 | 0.62 | 1.01 | 0.93 | 0.32 |
| 117C, 72A | 0.25 | −0.17 | −0.13 | −0.19 | −0.24 | −0.12 |
| 117D, 72A | 0.36 | 0.41 | 0.44 | 0.44 | 0.35 | 0.17 |
| 117E, 72A | 0.20 | 0.65 | 0.64 | 0.76 | 0.65 | 0.40 |
| 117F, 72A | 0.19 | 1.33 | 1.38 | 1.36 | 1.37 | 1.20 |
| 117G, 72A | 0.36 | 0.88 | 0.85 | 0.92 | 0.84 | 0.39 |
| 117H, 72A | 0.31 | 0.90 | 0.94 | 1.06 | 1.01 | 0.63 |
| 117I, 72A | 0.14 | 0.68 | 0.68 | 0.80 | 0.67 | 0.41 |
| 117K, 72A | 0.19 | 0.34 | 0.37 | 0.43 | 0.04 | 0.22 |
| 117L, 72A | 0.11 | 0.38 | 0.39 | 0.43 | 0.35 | 0.29 |
| 117M, 72A | 0.18 | 1.27 | 1.36 | 1.31 | 1.32 | 1.14 |
| 117N, 72A | 0.18 | 1.33 | 1.37 | 1.35 | 1.40 | 1.19 |
| 117P, 72A | 0.41 | 0.80 | 0.83 | 0.86 | 0.73 | 0.38 |
| 117Q, 72A | 0.37 | 1.21 | 1.24 | 1.37 | 1.13 | 0.63 |
| 117R, 72A | 0.41 | 0.64 | 0.70 | 0.70 | 0.61 | 0.31 |
| 117S, 72A | 0.36 | 1.35 | 1.07 | 1.42 | 1.19 | 0.55 |
| 117T, 72A | 0.20 | 0.79 | 0.77 | 0.92 | 0.81 | 0.57 |
| 117V, 72A | 0.16 | 0.82 | 0.81 | 0.94 | 0.81 | 0.49 |
| 117W, 72A | 0.29 | 0.55 | 0.46 | 0.45 | 0.39 | 0.34 |
| 117Y, 72A | 0.18 | 1.35 | 1.40 | 1.44 | 1.36 | 1.14 |
| 119A, 72A | 0.31 | 0.70 | 0.65 | 0.61 | 0.63 | 0.30 |
| 119C, 72A | 0.68 | 1.06 | 1.00 | 1.02 | 0.97 | 0.35 |
| 119F, 72A | 0.20 | 0.93 | 0.93 | 0.90 | 0.94 | 0.69 |
| 119G, 72A | 0.25 | 0.83 | 0.81 | 0.77 | 0.78 | 0.46 |
| 119H, 72A | 0.33 | 0.71 | 0.67 | 0.63 | 0.59 | 0.32 |
| 119I, 72A | 0.33 | 1.01 | 1.06 | 1.10 | 1.08 | 0.69 |
| 119K, 72A | 0.31 | 0.78 | 0.78 | 0.85 | 0.87 | 0.58 |
| 119Q, 72A | 0.52 | 0.60 | 0.62 | 0.58 | 0.60 | 0.35 |
| 119S, 72A | 0.31 | 0.78 | 0.73 | 0.46 | 0.71 | 0.37 |
| 119T, 72A | 0.25 | 1.22 | 1.21 | 1.22 | 1.26 | 0.79 |
| 119V, 72A | 0.33 | 0.99 | 1.00 | 1.01 | 1.02 | 0.57 |
| 11A1, L72A | 0.20 | 0.60 | 0.67 | 0.73 | 0.64 | 0.56 |
| 11C, 72A | 0.45 | 0.52 | 0.64 | 0.70 | 0.70 | 0.80 |
| 11C2, L72A | 0.49 | 0.51 | 0.67 | 0.67 | 0.66 | 0.55 |
| 11D1, L72A | 0.21 | 0.82 | 0.86 | 0.91 | 0.92 | 0.83 |
| 11E2, L72A | 0.24 | 0.54 | 0.52 | 0.62 | 0.55 | 0.54 |
| 11F1, L72A | 0.32 | 0.42 | 0.29 | 0.28 | 0.20 | 0.18 |
| 11G, 72A | 0.14 | 0.63 | 0.67 | 0.70 | 0.62 | 0.61 |
| 11G1, 72A | 0.17 | 0.81 | 0.81 | 0.92 | 0.85 | 0.78 |
| 11G1, | 0.14 | 0.55 | 0.59 | 0.70 | 0.60 | 0.53 |
| L72A | | | | | | |
| 11H1, 72A | 0.21 | 0.58 | 0.59 | 0.55 | 0.48 | 0.56 |
| 11H1, L72A | 0.18 | 0.48 | 0.50 | 0.47 | 0.39 | 0.48 |
| 11I1, L72A | 0.38 | 0.58 | 0.51 | 0.55 | 0.39 | 1.19 |
| 11L1, L72A | 0.39 | 0.47 | 0.35 | 0.30 | 0.15 | 0.27 |
| 11M2, 72A | 0.33 | 0.65 | 0.59 | 0.55 | 0.42 | 0.57 |
| 11M2, | 0.32 | 0.70 | 0.65 | 0.58 | 0.46 | 0.54 |
| L72A | | | | | | |
| 11Q1, 72A | 0.26 | 1.53 | 1.58 | 1.72 | 1.72 | 1.45 |
| 11Q1, | 0.21 | 1.33 | 1.43 | 1.37 | 1.32 | 1.19 |
| L72A | | | | | | |
| 11S5, 72A | 0.15 | 0.41 | 0.39 | 0.44 | 0.37 | 0.47 |
| 11S5, L72A | 0.12 | 0.33 | 0.37 | 0.36 | 0.29 | 0.35 |
| 11T1, 72A | 0.18 | 0.51 | 0.47 | 0.51 | 0.41 | 1.95 |
| 11T1, L72A | 0.17 | 0.49 | 0.47 | 0.45 | 0.35 | 0.35 |
| 11V2, 72A | 0.36 | 0.93 | 0.76 | 0.83 | 0.55 | 0.73 |
| 11W1, 72A | 0.26 | 0.43 | 0.36 | 0.34 | 0.23 | 0.20 |
| 11W1, | 0.29 | 0.43 | 0.39 | 0.40 | 0.22 | 0.15 |
| L72A | | | | | | |
| 11Y1, 72A | 0.31 | 0.52 | 0.31 | 0.33 | 0.16 | 0.30 |
| 11Y1, L72A | 0.33 | 0.49 | 0.33 | 0.32 | 0.16 | 0.25 |
| 126A, 72A | 0.19 | 1.27 | 1.36 | 1.32 | 1.38 | 1.22 |
| 126D, 72A | 0.38 | 0.44 | 0.56 | 0.59 | 0.64 | 0.37 |
| 126E, 72A | 0.33 | 0.25 | 0.32 | 0.34 | 0.30 | 0.21 |
| 126F, 72A | 0.38 | 0.52 | 0.53 | 0.51 | 0.43 | 0.34 |
| 126H, 72A | 0.31 | 0.20 | 0.46 | 0.46 | 0.43 | 0.32 |
| 126I, 72A | 0.30 | 0.45 | 0.43 | 0.43 | 0.34 | 0.38 |
| 126L, 72A | 0.34 | 0.66 | 0.66 | 0.64 | 0.55 | 0.38 |
| 126M, 72A | 0.32 | 0.32 | 0.28 | 0.31 | 0.24 | 0.29 |
| 126V, 72A | 0.28 | 0.42 | 0.42 | 0.45 | 0.33 | 0.34 |
| 14A, 72A | 0.44 | 0.53 | 0.56 | 0.56 | 0.36 | 0.24 |
| 14D, 72A | 0.44 | 0.46 | 0.51 | 0.53 | 0.34 | 0.28 |
| 14E, 72A | 0.41 | 0.51 | 0.61 | 0.56 | 0.41 | 0.44 |
| 14F, 72A | 0.22 | 0.83 | 0.88 | 0.88 | 0.72 | 0.60 |
| 14G, 72A | 0.44 | 0.43 | 0.46 | 0.42 | 0.24 | 0.19 |
| 14H, 72A | 0.35 | 0.37 | 0.42 | 0.45 | 0.25 | 0.17 |
| 14I, 72A | 0.39 | 0.57 | 0.90 | 0.79 | 0.57 | 0.59 |
| 14K, 72A | 0.36 | 1.08 | 0.92 | 1.06 | 0.84 | 0.47 |
| 14L, 72A | 0.31 | 0.89 | 0.93 | 0.87 | 0.44 | 3.07 |
| 14M, 72A | 0.33 | 0.62 | 0.68 | 0.68 | 0.41 | 0.45 |
| 14N, 72A | 0.45 | 0.42 | 0.53 | 0.49 | 0.35 | 0.32 |
| 14P, 72A | 0.46 | 0.55 | 0.64 | 0.64 | 0.58 | 0.30 |
| 14Q, 72A | 0.40 | 0.50 | 0.52 | 0.48 | 0.29 | 0.33 |
| 14R, 72A | 0.41 | 0.49 | 0.51 | 0.56 | 0.26 | 0.26 |
| 14S, 72A | 0.46 | 0.46 | 0.53 | 0.53 | 0.33 | 0.27 |
| 14T, 72A | 0.46 | 0.64 | 1.15 | 0.76 | 0.56 | 0.36 |
| 14V, 72A | 0.32 | 0.76 | 0.87 | 0.84 | 0.51 | 0.43 |
| 14W, 72A | 0.11 | 0.30 | 0.31 | 0.33 | 0.26 | 0.23 |
| 14Y, 72A | 0.17 | 1.29 | 1.29 | 1.23 | 2.22 | 1.07 |
| wt | | | | | | |
| 17A, 72A | 0.28 | 0.53 | 0.97 | 0.56 | 0.38 | 0.24 |
| 17C, 72A | 0.36 | 0.15 | 0.26 | 0.25 | 0.31 | 0.10 |
| 17D, 72A | 0.40 | 0.28 | 0.34 | 0.31 | 0.26 | 0.16 |
| 17E, 72A | 0.27 | 0.80 | 0.88 | 0.90 | 0.84 | 0.53 |
| 17F, 72A | 0.19 | 1.37 | 1.38 | 1.35 | 1.42 | 1.14 |
| 17G, 72A | 0.39 | 0.48 | 0.62 | 0.58 | 0.52 | 0.28 |
| 17H, 72A | 0.36 | 0.52 | 0.50 | 0.48 | 0.40 | 0.22 |
| 17I, 72A | 0.26 | 0.64 | 0.54 | 0.50 | 0.62 | 0.28 |
| 17K 72A | 0.41 | 0.28 | 0.29 | 0.33 | 0.31 | 0.13 |
| 17L, 72A | 0.25 | 0.52 | 0.45 | 0.42 | 0.46 | 0.28 |
| 17M, 72A | 0.30 | 0.59 | 0.53 | 0.49 | 0.49 | 0.28 |
| 17N, 72A | 0.34 | 0.47 | 0.51 | 0.50 | 0.49 | 0.22 |
| 17P, 72A | 0.45 | 0.51 | 0.63 | 0.60 | 0.55 | 0.24 |
| 17Q, 72A | 0.25 | 0.62 | 0.59 | 0.59 | 0.55 | 0.29 |
| 17R, 72A | 0.36 | 0.43 | 0.48 | 0.52 | 0.45 | 0.22 |
| 17S, 72A | 0.35 | 0.50 | 0.61 | 0.56 | 0.47 | 0.25 |
| 17T, 72A | 0.32 | 0.50 | 0.51 | 0.51 | 0.49 | 0.25 |
| 17V, 72A | 0.30 | 0.64 | 0.59 | 0.55 | 0.56 | 0.31 |
| 17W, 72A | 0.18 | 1.42 | 1.61 | 1.42 | 1.24 | 1.07 |
| 17Y, 72A | 0.65 | 0.73 | 0.81 | 0.71 | 0.73 | 0.50 |
| 18Q, 72A | 0.22 | 0.09 | 0.13 | 0.11 | 0.08 | 0.09 |
| 21W, 72A | 0.60 | 3.70 | 2.40 | 2.43 | 3.02 | 1.45 |
| 23F2, 72A | 0.22 | 0.22 | 0.17 | 0.19 | 0.20 | 0.12 |
| 23H1, 72A | 0.20 | 0.16 | 0.17 | 0.13 | 0.11 | 0.13 |
| 23K1, 72A | 0.45 | 0.13 | 0.13 | 0.14 | 0.13 | 0.08 |
| 23L3, 72A | 0.23 | 1.17 | 1.03 | 1.04 | 1.06 | 0.93 |
| 23N1, 72A | 0.22 | 0.12 | 0.13 | 0.13 | 0.12 | 0.10 |
| 23P1, 72A | 0.32 | 1.25 | 1.38 | 1.36 | 1.24 | 0.64 |
| 23R3, 72A | 0.20 | 0.14 | 0.14 | 0.14 | 0.12 | 0.10 |
| 23T1, 72A | 0.16 | 0.22 | 0.21 | 0.33 | 0.19 | 0.24 |
| 23V2, 72A | 0.17 | 1.46 | 1.35 | 1.73 | 1.56 | 1.48 |
| 23W1, 72A | 0.45 | 0.62 | 0.96 | 0.79 | 0.45 | 0.49 |
| 23Y1, 72A | 0.26 | 0.49 | 0.44 | 0.44 | 0.41 | 0.28 |
| 31C1, 72A | 0.30 | 0.32 | 0.36 | 0.36 | 0.30 | 0.23 |
| 31D2, 72A | 0.31 | 0.20 | 0.14 | 0.16 | 0.13 | 0.06 |
| 31E3, 72A | 0.35 | 0.55 | 0.82 | 0.74 | 0.50 | 0.38 |
| 31F2, 72A | 0.43 | 1.41 | 1.62 | 1.76 | 1.43 | 1.21 |
| 31I1, 72A | 0.68 | 0.30 | 0.41 | 0.17 | −0.21 | 0.09 |
| 31K2, 72A | 0.26 | 0.85 | 0.90 | 0.91 | 0.97 | 0.42 |
| 31M2, 72A | 0.81 | 0.52 | 0.57 | 0.41 | 1.08 | 0.46 |
| 31N2, 72A | 0.36 | 0.88 | 1.06 | 1.04 | 0.91 | 0.50 |
| 31P1, 72A | 0.27 | 0.25 | 0.27 | 0.30 | 0.22 | 0.23 |
| 31Q3, 72A | 0.38 | 1.02 | 1.49 | 1.47 | 1.31 | 1.04 |
| 31R1, 72A | 0.18 | 0.25 | 0.44 | 0.40 | 0.23 | 0.34 |
| 31T2, 72A | 0.37 | 0.65 | 0.91 | 0.75 | 0.53 | 0.41 |
| 31V2, 72A | 0.61 | 0.40 | 0.78 | 0.38 | 0.08 | 0.21 |
| 31W2, 72A | 0.46 | 0.99 | 1.03 | 1.15 | 0.87 | 0.62 |
| 31Y1, 72A | 0.48 | 1.78 | 1.61 | 1.75 | 1.55 | 1.10 |
| 31Y2, 72A | 0.69 | 0.37 | 0.80 | 0.36 | 0.01 | 0.17 |
| 34A, 72A | 0.35 | 0.58 | 0.57 | 0.57 | 0.49 | 0.33 |
| 34C, 72A | 0.44 | 0.34 | 0.33 | 0.59 | 0.30 | 0.18 |
| 34E, 72A | 0.32 | 0.62 | 0.57 | 0.60 | 0.49 | 0.48 |
| 34G, 72A | 0.51 | 0.54 | 0.29 | 0.33 | 0.13 | 0.18 |
| 34H, 72A | 0.37 | 0.65 | 0.72 | 0.74 | 0.52 | 0.38 |
| 34K, 72A | 0.48 | 0.43 | 1.33 | 0.61 | 0.41 | 0.26 |
| 34N, 72A | 0.16 | 1.19 | 1.18 | 1.16 | 1.16 | 1.11 |
| 34P, 72A | 0.42 | 0.46 | 0.76 | 0.66 | 0.46 | 0.35 |
| 34Q, 72A | 0.37 | 0.52 | 0.48 | 0.56 | 0.44 | 0.36 |
| 34R, 72A | 0.52 | 0.41 | 0.47 | 0.61 | 0.44 | 0.30 |
| 34S, 72A | 0.36 | 0.58 | 0.59 | 0.65 | 0.58 | 0.43 |
| 34T, 72A | 0.34 | 0.61 | 0.67 | 0.64 | 0.53 | 0.40 |
| 34V, 72A | 0.17 | 1.22 | 1.21 | 1.15 | 1.19 | 1.11 |
| 34W, 72A | 0.24 | 1.31 | 1.37 | 1.34 | 1.38 | 0.93 |
| 34Y, 72A | 0.18 | 1.22 | 1.22 | 1.24 | 1.19 | 1.06 |
| 36A, 72A | 0.27 | 0.53 | 0.50 | 0.51 | 0.46 | 0.46 |
| 36C3, 72A | 0.38 | −0.22 | −0.30 | −0.36 | −0.40 | −0.21 |
| 36D1, 72A | 0.42 | 0.20 | 0.21 | 0.27 | 0.20 | 0.18 |
| 36E2, 72A | 0.31 | 0.24 | 0.23 | 0.25 | 0.20 | 0.19 |
| 36F1, 72A | 0.25 | 0.66 | 0.66 | 0.67 | 0.55 | 0.52 |
| 36G2, 72A | 0.36 | 1.00 | 1.16 | 1.17 | 0.97 | 0.55 |
| 36H1, 72A | 0.34 | 0.76 | 0.86 | 0.81 | 0.77 | 0.40 |
| 36I1, 72A | 0.29 | 1.01 | 1.13 | 1.11 | 0.95 | 0.80 |
| 36K, 72A | 0.43 | 0.38 | 0.42 | 0.38 | 0.41 | 0.16 |
| 36L, 72A wt | 0.16 | 1.25 | 1.00 | 1.21 | 1.25 | 1.13 |
| 36M, 72A | 0.20 | 1.23 | 1.08 | 1.10 | 1.12 | 0.97 |
| 36N, 72A | 0.35 | 0.47 | 0.42 | 0.47 | 0.38 | 0.39 |
| 36P, 72A | 0.44 | 0.32 | 0.36 | −7.45 | 0.14 | 0.31 |
| 36Q, 72A | 0.30 | 1.20 | 1.28 | 1.26 | 1.12 | 0.81 |
| 36R, 72A | 0.30 | 1.04 | 1.17 | 1.17 | 1.14 | 0.70 |
| 36S, 72A | 0.31 | 0.49 | 0.50 | 0.50 | 0.43 | 0.42 |
| 36Y, 72A | 0.24 | 0.68 | 0.68 | 0.75 | 0.66 | 0.60 |
| 38A2, 72A | 0.32 | 1.23 | 1.30 | 1.31 | 1.26 | 0.68 |
| 38E1, 72A | 0.24 | 0.57 | 0.29 | 0.59 | 0.54 | 0.31 |
| 38F1, 72A | 0.19 | 0.91 | 0.96 | 0.95 | 0.97 | 0.85 |
| 38G1, 72A | 0.34 | 1.03 | 1.07 | 1.10 | 1.03 | 0.56 |
| 38H1, 72A | 0.27 | 1.24 | 1.19 | 1.25 | 1.16 | 0.74 |
| 38I3, 72A | 0.19 | 1.18 | 1.21 | 1.22 | 1.21 | 1.12 |
| 38K1, 72A | 0.26 | 0.83 | 0.86 | 0.90 | 0.76 | 0.48 |
| 38N1, 72A | 0.50 | 1.08 | 1.14 | 1.12 | 0.97 | 0.65 |
| 38Q1, 72A | 0.26 | 1.87 | 1.87 | 1.90 | 1.75 | 1.30 |
| 38S1, 72A | 0.32 | 1.28 | 1.37 | 1.37 | 1.28 | 0.71 |
| 38T3, 72A | 0.30 | 1.60 | 1.64 | 1.58 | 1.55 | 1.02 |
| 38V2, 72A | 0.21 | 1.65 | 1.67 | 1.70 | 1.75 | 1.50 |
| 38W1, 72A | 0.23 | 1.35 | 1.34 | 2.41 | 1.58 | 1.55 |
| 38Y1, 72A | 0.21 | 2.08 | 1.88 | 1.82 | 1.72 | 1.52 |
| 40F2, L72A | 0.20 | 0.89 | 0.99 | 0.90 | 0.90 | 0.80 |
| 40M1, | 0.20 | 0.99 | 1.02 | 1.09 | 0.99 | 0.69 |
| L72A | | | | | | |
| 40V1, L72A | 0.21 | 1.20 | 1.28 | 1.24 | 1.21 | 0.91 |
| 40Y3, L72A | 0.22 | 0.89 | 0.49 | 0.50 | 0.48 | 0.29 |
| 47A1, L72A | 0.29 | 0.54 | 0.57 | 0.58 | 0.59 | 0.30 |
| 47C3, L72A | 0.24 | 0.32 | 0.35 | 0.37 | 0.35 | 0.23 |
| 47E3, L72A | 0.18 | 1.25 | 1.26 | 1.23 | 1.25 | 1.06 |
| 47G1, | 0.34 | 0.51 | 0.63 | 0.66 | 0.59 | 0.32 |
| L72A | | | | | | |
| 47H1, L72A | 0.22 | 0.63 | 0.70 | 0.74 | 0.67 | 0.43 |
| 47I2, L72A | 0.19 | 1.19 | 1.19 | 1.22 | 1.15 | 0.92 |
| 47L2, L72A | 0.22 | 1.15 | 1.20 | 1.21 | 1.20 | 0.90 |
| 47M, 72A | 0.23 | 1.14 | 1.13 | 1.18 | 1.17 | 0.97 |
| 47M2, | 0.18 | 1.00 | 0.97 | 0.99 | 0.95 | 0.83 |
| L72A | | | | | | |
| 47P1, L72A | 0.36 | 0.54 | 0.61 | 0.59 | 0.57 | 0.26 |
| 47Q3, | 0.16 | 0.72 | 0.73 | 0.78 | 0.75 | 0.46 |
| L72A | | | | | | |
| 47R3, L72A | 0.33 | 0.56 | 0.61 | 0.64 | 0.62 | 0.30 |
| 47S1, L72A | 0.31 | 0.61 | 0.69 | 0.66 | 0.64 | 0.34 |
| 47T1, L72A | 0.24 | 0.68 | 0.74 | 0.77 | 0.74 | 0.46 |
| 47V3, L72A | 0.19 | 1.00 | 1.01 | 1.04 | 1.01 | 0.78 |
| 47W1, | 0.17 | 1.18 | 1.14 | 1.18 | 1.22 | 1.07 |
| L72A | | | | | | |
| 47Y2, L72A | 0.19 | 1.37 | 1.40 | 1.40 | 1.42 | 1.17 |
| 49A1, 72A | 0.45 | 1.72 | 1.68 | 1.72 | 1.60 | 0.79 |
| 49C1, 72A | 0.29 | −0.13 | −0.18 | −0.21 | −0.20 | −0.18 |
| 49D1, 72A | 0.23 | 0.31 | 0.30 | 0.32 | 0.32 | 0.16 |
| 49F1, 72A | 0.39 | 1.18 | 1.18 | 1.14 | 1.14 | 0.75 |
| 49G1, 72A | 0.43 | 1.26 | 1.25 | 1.29 | 1.15 | 0.64 |
| 49H1, 72A | 0.44 | 1.47 | 1.54 | 1.58 | 1.39 | 0.75 |
| 49I1, 72A | 0.36 | 1.80 | 1.82 | 1.84 | 1.80 | 1.16 |
| 49K1, 72A | 0.43 | 1.64 | 1.68 | 1.71 | 1.51 | 0.80 |
| 49L1, 72A | 0.37 | 1.76 | 1.77 | 1.81 | 1.67 | 1.00 |
| 49M1, 72A | 0.35 | 1.36 | 1.37 | 1.37 | 1.30 | 0.94 |
| 49N1, 72A | 0.35 | 1.26 | 1.18 | 1.10 | 1.11 | 0.74 |
| 49P1, 72A | 0.40 | 1.57 | 1.53 | 1.59 | 1.40 | 0.76 |
| 49Q, 72A | 0.23 | 0.62 | 0.55 | 0.58 | 0.56 | 0.43 |
| 49Q1, 72A | 0.39 | 0.92 | 1.23 | 1.25 | 1.20 | 0.69 |
| 49R1, 72A | 0.42 | 1.68 | 1.70 | 1.75 | 1.56 | 0.78 |
| 49S1, 72A | 0.40 | 1.27 | 1.34 | 1.29 | 1.18 | 0.66 |
| 49T1, 72A | 0.28 | 0.66 | 0.64 | 0.67 | 0.67 | 0.43 |
| 49W1, 72A | 0.31 | 0.58 | 0.58 | 0.58 | 0.56 | 0.35 |
| 49Y1, 72A | 0.43 | 1.39 | 1.44 | 1.51 | 1.34 | 0.70 |
| 51A1, 72A | 0.41 | 1.38 | 1.49 | 1.48 | 1.28 | 0.70 |
| 51C3, 72A | 0.42 | 1.44 | 1.47 | 1.55 | 1.35 | 0.69 |
| 51D1, 72A | 0.42 | 1.34 | 1.39 | 1.38 | 1.28 | 0.68 |
| 51F1, 72A | 0.43 | 1.64 | 1.60 | 1.70 | 1.47 | 0.76 |
| 51G1, 72A | 0.41 | 1.64 | 1.76 | 1.71 | 1.57 | 0.78 |
| 51H1, 72A | 0.35 | 1.52 | 1.58 | 1.64 | 0.58 | 0.81 |
| 51I1, 72A | 0.39 | 1.50 | 1.62 | 1.55 | 1.41 | 0.72 |
| 51K1, 72A | 0.37 | 1.00 | 1.02 | −0.01 | 1.01 | 0.56 |
| 51L3, 72A | 0.39 | 1.64 | 1.66 | 1.65 | 1.45 | 0.76 |
| 51N1, 72A | 0.38 | 1.38 | 1.39 | 1.46 | 1.26 | 0.73 |
| 51P3, 72A | 0.38 | 1.42 | 1.48 | 1.42 | 1.21 | 0.72 |
| 51Q1, 72A | 0.40 | 1.56 | 1.65 | 1.63 | 1.43 | 0.73 |
| 51R1, 72A | 0.40 | 1.50 | 1.56 | 1.56 | 1.35 | 0.73 |
| 51S1, 72A | 0.38 | 1.64 | 1.66 | 1.78 | 1.61 | 0.80 |
| 51T3, 72A | 0.40 | 1.60 | 1.60 | 1.65 | 1.44 | 0.71 |
| 51V3, 72A | 0.40 | 1.52 | 1.53 | 1.64 | 1.43 | 0.73 |
| 51W2, 72A | 0.39 | 1.69 | 1.70 | 1.77 | 1.55 | 0.80 |
| 51Y1, 72A | 0.38 | 1.38 | 1.44 | 1.50 | 1.38 | 0.72 |
| 55A1, 72A | 0.18 | 0.33 | 0.36 | 0.34 | 0.30 | 0.28 |
| 55D1, 72A | 0.18 | 0.11 | 0.12 | 0.12 | 0.10 | 0.10 |
| 55E, 72A | 0.25 | 0.21 | 0.21 | 0.21 | 0.21 | 0.17 |
| 55G1, 72A | 0.20 | 0.19 | 0.23 | 0.22 | 0.19 | 0.18 |
| 55H2, 72A | 0.18 | 0.36 | 0.32 | 0.38 | 0.27 | 0.26 |
| 55I1, 72A | 0.18 | 1.08 | 0.89 | 0.83 | 0.90 | 0.77 |
| 55K1, 72A | 0.30 | 0.32 | 0.35 | 0.34 | 0.30 | 0.27 |
| 55L1, 72A | 0.16 | 0.86 | 0.72 | 0.71 | 0.82 | 0.63 |
| 55M1, 72A | 0.36 | 1.40 | 1.42 | 0.97 | 1.41 | 0.84 |
| 55N1, 72A | 0.20 | 0.34 | 0.30 | 0.29 | 0.27 | 0.22 |
| 55P1, 72A | 0.33 | 1.01 | 0.96 | 1.05 | 0.93 | 0.54 |
| 55Q1, 72A | 0.23 | 0.60 | 0.49 | 0.47 | 0.54 | 0.43 |
| 55R1, 72A | 0.24 | 0.40 | 0.37 | 0.41 | 0.39 | 0.30 |
| 55S1, 72A | 0.20 | 0.48 | 0.43 | 0.42 | 0.41 | 0.41 |
| 55T1, 72A | 0.17 | 1.26 | 1.27 | 1.25 | 1.28 | 1.12 |
| 55W2, 72A | 0.23 | 0.77 | 0.82 | 0.79 | 0.70 | 0.63 |
| 55Y3, 72A | 0.12 | 0.47 | 0.34 | 0.32 | 0.33 | 0.25 |
| 60A, 72A | 0.23 | 0.79 | 0.94 | 0.98 | 0.94 | 0.74 |
| 60C, 72A | 0.17 | 1.30 | 1.36 | 1.33 | 1.34 | 1.04 |
| 60F, 72A | 0.25 | 1.79 | 1.76 | 1.68 | 1.90 | 1.53 |
| 60G, 72A | 0.23 | 0.29 | 0.24 | 0.25 | 0.24 | 0.19 |
| 60H, 72A | 0.29 | 0.17 | 0.18 | 0.17 | 0.17 | 0.12 |
| 60I, 72A | 0.20 | 1.54 | 1.63 | 1.73 | 1.72 | 1.52 |
| 60K, 72A | 0.41 | 0.46 | 0.57 | 0.62 | 0.60 | 0.35 |
| 60L, 72A | 0.24 | 1.08 | 1.26 | 1.29 | 1.27 | 1.10 |
| 60M, 72A | 0.29 | 0.77 | 0.93 | 1.01 | 0.91 | 0.76 |
| 60N, 72A | 0.21 | 1.24 | 1.59 | 1.57 | 1.54 | 1.30 |
| 60P, 72A | 0.40 | 0.62 | 0.77 | 0.81 | 0.81 | 0.37 |
| 60Q, 72A | 0.27 | 0.36 | 0.42 | 0.41 | 0.41 | 0.35 |
| 60R, 72A | 0.27 | 0.14 | 0.14 | 0.12 | 0.13 | 0.08 |
| 60S, 72A | 0.26 | 0.56 | 0.71 | 0.72 | 0.67 | 0.59 |
| 60T, 72A | 0.22 | 1.72 | 1.73 | 1.73 | 1.76 | 1.49 |
| 60W, 72A | 0.18 | 0.67 | 0.74 | 0.84 | 0.76 | 0.59 |
| 62A, 72A | 0.27 | 1.08 | 1.08 | 0.96 | 0.86 | 0.57 |
| 62C, 72A | 0.19 | 1.30 | 1.31 | 1.31 | 1.29 | 1.18 |
| 62D, 72A | 0.23 | 1.49 | 1.58 | 1.60 | 1.57 | 1.39 |
| 62E, 72A | 0.21 | 1.48 | 1.52 | 1.49 | 1.52 | 1.40 |
| 62G, 72A | 0.22 | 1.43 | 1.44 | 1.44 | 1.46 | 1.26 |
| 62H, 72A | 0.20 | 1.42 | 1.44 | 1.40 | 1.40 | 1.24 |
| 62I, 72A | 0.19 | 1.40 | 1.38 | 1.36 | 1.37 | 1.27 |
| 62N, 72A | 0.20 | 1.40 | 1.40 | 1.34 | 1.37 | 1.29 |
| 62T, 72A | 0.24 | 0.99 | 0.96 | 1.02 | 1.00 | 0.65 |
| 68A, 72A | 0.23 | 0.23 | 0.19 | 0.18 | 0.19 | 0.13 |
| 68C, 72A | 0.58 | 0.18 | 0.25 | 0.12 | 0.29 | 0.13 |
| 68D, 72A | 0.38 | 1.24 | 1.23 | 1.23 | 1.13 | 0.52 |
| 68E, 72A | 0.36 | 0.89 | 0.92 | 0.96 | 0.91 | 0.53 |
| 68G, 72A | 0.23 | 0.24 | 0.18 | 0.17 | 0.21 | 0.12 |
| 68I, 72A | 0.22 | 0.42 | 0.36 | 0.37 | 0.39 | 0.35 |
| 68L, 72A | 0.21 | 0.66 | 0.52 | 0.53 | 0.55 | 0.52 |
| 68M, 72A | 0.19 | 0.52 | 0.35 | 0.34 | 0.35 | 0.33 |
| 68N, 72A | 0.33 | 0.67 | 0.60 | 0.67 | 0.63 | 0.35 |
| 68Q, 72A | 0.30 | 0.47 | 0.38 | 0.43 | 0.45 | 0.28 |
| 68S, 72A | 0.21 | 0.20 | 0.14 | 0.14 | 0.14 | 0.10 |
| 68T, 72A | 0.18 | 0.18 | 0.12 | 0.11 | 0.13 | 0.10 |
| 68V, 72A | 0.20 | 0.29 | 0.22 | 0.23 | 0.25 | 0.19 |
| 68Y, 72A | 0.17 | 0.42 | 0.41 | 0.36 | 0.46 | 0.35 |
| 70C, 72A | 0.43 | 0.23 | 0.24 | 0.27 | 0.24 | 0.14 |
| 70M, 72A | 0.27 | 0.34 | 0.53 | 0.39 | 0.42 | 0.26 |
| 72C | 0.30 | 0.14 | 0.17 | 0.25 | 0.20 | 0.12 |
| 72D | 0.47 | 0.27 | 0.41 | 0.37 | 0.28 | 0.24 |
| 72E | 0.57 | 0.27 | 0.36 | 0.38 | 0.34 | 0.19 |
| 72F | 0.52 | 0.40 | 0.59 | 0.61 | 0.57 | 0.46 |
| 72G | 0.28 | 0.62 | 0.48 | 0.48 | 0.52 | 0.26 |
| 72K | 0.40 | 0.26 | 0.31 | 0.26 | 0.19 | 0.13 |
| 72M | 0.38 | 1.31 | 1.35 | 1.46 | 1.49 | 1.32 |
| 72N | 0.31 | 0.65 | 0.70 | 0.77 | 0.83 | 0.44 |
| 72Q | 0.66 | 0.77 | 0.73 | 0.76 | 0.78 | 0.99 |
| 72R | 0.69 | 0.45 | 0.49 | 0.51 | 0.45 | 0.26 |
| 72S | 0.21 | 0.65 | 0.71 | 0.73 | 0.62 | 0.68 |
| 72V | 0.09 | 0.18 | 0.15 | 0.21 | 0.20 | 0.16 |
| 72Y | 0.39 | 0.35 | 0.59 | 0.64 | 0.56 | 0.41 |
| 73E, 72A | 0.23 | 0.15 | 0.14 | 0.19 | 0.14 | 0.13 |
| 73F, 72A | 0.33 | 1.21 | 1.86 | 1.23 | 0.73 | 1.13 |
| 73G, 72A | 0.22 | 0.46 | 0.53 | 0.56 | 0.52 | 0.41 |
| 73H, 72A | 0.11 | 0.15 | 0.25 | 0.18 | 0.13 | 0.16 |
| 73K, 72A | 0.24 | 0.30 | 0.28 | 0.29 | 0.28 | 0.15 |
| 73L, 72A | 0.29 | 0.73 | 0.34 | 0.33 | 0.38 | 0.25 |
| 73N, 72A | 0.21 | 0.48 | 0.47 | 0.43 | 0.41 | 0.39 |
| 73P, 72A | 0.15 | 0.42 | 0.41 | 0.30 | 0.30 | 0.26 |
| 73Q, 72A | 0.27 | 1.36 | 1.31 | 1.39 | 1.28 | 1.19 |
| 73R, 72A | 0.27 | 0.40 | 0.33 | 0.36 | 0.27 | 0.20 |
| 73S, 72A | 0.20 | 1.01 | 1.08 | 1.05 | 0.96 | 0.99 |
| 73T, 72A | 0.16 | 0.87 | 0.77 | 0.77 | 0.78 | 0.62 |
| 73V, 72A | 0.30 | 0.98 | 0.50 | 0.40 | 0.60 | 0.48 |
| 73W, 72A | 0.39 | 1.77 | 1.93 | 1.89 | 1.66 | 1.05 |
| 74A, 72A | 0.20 | 1.42 | 1.42 | 1.34 | 1.34 | 1.27 |
| 74C, 72A | 0.45 | 0.83 | 0.74 | 0.71 | 0.56 | 0.29 |
| 74F, 72A | 0.25 | 1.02 | 1.36 | 1.19 | 1.10 | 1.23 |
| 74L, 72A | 0.17 | 0.22 | 0.40 | 0.30 | 0.19 | 0.36 |
| 74N, 72A | 0.16 | 1.62 | 1.57 | 1.44 | 1.52 | 1.38 |
| 74Q, 72A | 0.29 | 0.81 | 0.86 | 0.87 | 0.74 | 0.43 |
| 74T, 72A | 0.27 | 0.76 | 0.78 | 0.82 | 0.70 | 0.37 |
| 78A3, 72A | 0.25 | 0.48 | 0.50 | 0.44 | 0.41 | 0.25 |
| 78C2, 72A | 0.35 | 0.15 | 0.20 | 0.20 | 0.19 | 0.09 |
| 78F1, 72A | 0.35 | 2.04 | 2.05 | 2.00 | 1.83 | 0.91 |
| 78H1, 72A | 0.39 | 1.23 | 1.26 | 1.28 | 1.13 | 0.57 |
| 78I1, 72A | 0.56 | 1.27 | 1.66 | 1.11 | 0.93 | 0.52 |
| 78M1, 72A | 0.33 | 1.39 | 1.50 | 1.44 | 1.44 | 0.92 |
| 78T1, 72A | 0.40 | 1.07 | 1.20 | 1.25 | 1.08 | 0.54 |
| 78W1, 72A | 0.32 | 0.49 | 0.53 | 0.57 | 0.53 | 0.29 |
| 82A, 106A, | 0.29 | 0.11 | 0.13 | 0.18 | 0.13 | 0.05 |
| 72A | | | | | | |
| 82A, 72A | 0.41 | 1.55 | 1.56 | 1.67 | 1.53 | 0.84 |
| 82A1, 72A | 0.31 | 0.73 | 0.66 | 0.78 | 0.80 | 0.26 |
| 82F1, 72A | 0.27 | 0.68 | 0.76 | 1.08 | 0.78 | 0.41 |
| 82I3, 72A | 0.24 | 0.67 | 0.45 | 0.52 | 0.54 | 0.36 |
| 82M1, 72A | 0.27 | 0.97 | 0.75 | 0.94 | 0.92 | 0.54 |
| 82V1, 72A | 0.33 | 0.86 | 0.76 | 0.74 | 0.65 | 0.48 |
| 82Y2, 72A | 0.33 | 0.65 | 1.57 | 1.53 | 1.53 | 0.88 |
| 91A, 72A | 0.21 | 0.86 | 0.91 | 0.83 | 0.85 | 0.80 |
| 93L, 72A | 0.41 | 0.36 | 0.81 | 1.03 | 1.05 | 0.62 |
| 93M, 72A | 0.33 | 0.93 | 1.19 | 1.28 | 1.24 | 0.75 |

AA denotes Arachidonic acid;
LNA denotes linolenic acid;
LA denotes linoleic acid;
OA denotes oleic acid;
PA denotes palmitic acid

TABLE 2
Substitutions are shown with respect to the sequence of SEQ ID NO: 2.

| | | DR/DR | DR/DR | DR/DR | DR/DR | DR/DR | DR/DR | DR/DR | |
|---|---|---|---|---|---|---|---|---|---|
| Mutations | Ro | AA | LNA | LA | OA | PA | POA | SA | |
| 21W 72A | 0.616 | 3.829 | 2.550 | 2.551 | 2.808 | 1.637 | 2.120 | 1.946 | Large Response, Favors AA |
| 21W 78I 72A | 0.664 | 6.322 | 4.024 | 4.381 | 4.703 | 2.759 | 3.284 | 3.396 | |
| 21W 78F 72A | 0.604 | 4.264 | 1.694 | 1.597 | 1.843 | 0.851 | 1.217 | 1.161 | |
| 21W 78A 102V 72A | 0.388 | 4.923 | 1.628 | 2.749 | 3.261 | 1.721 | 1.585 | 3.092 | |
| 21F 78I 102F 72A | 0.400 | 4.836 | 3.006 | 2.872 | 3.312 | 2.383 | 2.788 | 2.367 | |
| 21F 78V 102V 72A | 0.288 | 4.511 | 3.775 | 4.204 | 4.223 | 2.756 | 3.365 | 3.120 | |
| 21Y 78I 102I 72A | 0.291 | 3.536 | 2.454 | 2.879 | 3.424 | 1.870 | 2.089 | 2.774 | |
| 21Y, 72A | 0.432 | 2.378 | 1.881 | 2.015 | 1.982 | 1.094 | | | |
| 38Y 62W 117A 72A | 0.554 | 2.353 | 1.237 | 1.848 | 1.896 | 0.316 | 0.272 | 0.854 | Favors AA |
| 72S 73Q 74A | 0.295 | 2.290 | 2.077 | 2.140 | 1.875 | 1.896 | 2.293 | 1.311 | |
| 38Y 117A 72A | 0.350 | 2.216 | 1.547 | 1.869 | 1.976 | 0.933 | 0.839 | 1.201 | |
| 38Y, 72A | 0.213 | 2.081 | 1.884 | 1.818 | 1.717 | 1.518 | | | |
| 14M 18L 31W 73I 117G 72A | 0.580 | 2.032 | 1.869 | 2.034 | 1.712 | 0.998 | 1.569 | 0.487 | |
| 38H 62W 106V 117A 72A | 0.284 | 2.007 | 1.796 | 1.337 | 1.239 | 1.002 | 1.145 | 0.947 | |
| 14L 31W 117V 72A | 0.259 | 1.972 | 1.894 | 2.027 | 1.580 | 1.304 | 1.768 | 0.742 | |
| 38M, 72A | 0.261 | 1.850 | 1.697 | 1.805 | 1.630 | 1.131 | | | |
| 18G 31F 73L 72A | 0.225 | 1.785 | 1.235 | 1.520 | 1.365 | 1.025 | 1.235 | 1.690 | |
| 38M 104S 115A 74F 72A | 0.325 | 1.690 | 1.615 | 1.778 | 1.155 | 0.498 | 1.172 | 0.356 | |
| 38Q 62I 106I 117A 72A | 0.215 | 1.656 | 0.930 | 1.288 | 1.167 | 0.550 | 0.473 | 0.663 | |
| 62Y, 72A | 0.238 | 1.598 | 1.395 | 1.545 | 1.439 | 1.037 | | | |
| 38Y 72G 117M | 0.204 | 1.541 | 1.228 | 1.233 | 1.137 | 0.574 | | | |
| 38A 106V 117A 72A | 0.418 | 1.492 | 0.541 | 0.229 | 0.368 | 0.033 | 0.121 | 0.065 | |
| 115W, 72A | 0.367 | 1.483 | 1.396 | 1.486 | 1.352 | 0.805 | | | |
| 72A 117A | 0.273 | 1.480 | 0.689 | 1.193 | 1.080 | 0.433 | | | |
| 117S, 72A | 0.357 | 1.467 | 1.133 | 1.533 | 1.258 | 0.593 | | | |
| 117A, 72A | 0.270 | 1.332 | 0.631 | 1.045 | 0.947 | 0.331 | | | |
| 14L 18I 31L 73F 117A 72A | 0.255 | 1.297 | 0.639 | 0.707 | 0.300 | 0.121 | 0.235 | 0.077 | |
| 38M 72G 117F | 0.241 | 1.280 | 0.733 | 0.748 | 0.686 | 0.438 | | | |
| 62N 106F 117A 72A | 0.224 | 1.265 | 0.756 | 0.985 | 0.864 | 0.430 | 0.341 | 0.490 | |
| 49N, 72A | 0.345 | 1.255 | 1.181 | 1.099 | 1.108 | 0.740 | | | |
| 14L 18T 31E 73G 117G 72A | 0.395 | 1.236 | 1.133 | 1.107 | 1.096 | 0.712 | 1.072 | 0.413 | |
| 14L 18L 31L 73G 117V 72A | 0.440 | 1.180 | 1.139 | 1.170 | 0.031 | 0.377 | 0.180 | 0.170 | |
| 23L, 72A | 0.229 | 1.174 | 1.025 | 1.041 | 1.057 | 0.928 | | | |
| 78Y, 72A | 0.314 | 1.172 | 0.871 | 0.908 | 0.845 | 0.520 | | | |
| 78I, 72A | 0.176 | 1.138 | 0.958 | 0.874 | 0.923 | 0.748 | | | |
| 106W, 72A | 0.241 | 1.106 | 1.030 | 1.042 | 0.991 | 0.407 | | | |
| 78V, 72A | 0.255 | 1.093 | 0.798 | 0.865 | 0.864 | 0.615 | | | |
| 55I, 72A | 0.181 | 1.079 | 0.885 | 0.834 | 0.901 | 0.774 | | | |
| 38Q 106W 117A 72A | 0.301 | 1.078 | 0.795 | 0.860 | 0.899 | 0.444 | 0.970 | 0.281 | |
| 104R, 72A | 0.436 | 1.075 | 0.904 | 0.962 | 0.957 | 0.791 | | | |
| 119C, 72A | 0.679 | 1.063 | 1.001 | 1.022 | 0.971 | 0.355 | | | |
| 82F, 72A | 0.139 | 1.029 | 0.334 | 0.422 | 0.403 | 0.223 | | | |
| 104N, 72A | 0.396 | 1.018 | 0.391 | 0.385 | 0.408 | 0.406 | | | |
| 38H, 72A | 0.204 | 0.998 | 0.864 | 0.905 | 0.955 | 0.624 | | | |
| 73V, 72A | 0.300 | 0.976 | 0.496 | 0.396 | 0.603 | 0.475 | | | |
| 38I 72G | 0.290 | 0.964 | 0.701 | 0.772 | 0.861 | 0.419 | | | |
| 14L 18S 31L 73V 117A 72A | 0.486 | 0.951 | 0.673 | 0.534 | 0.020 | 0.028 | −0.003 | 0.007 | |
| 21V, 72A | 0.269 | 0.939 | 0.889 | 0.871 | 0.739 | 0.462 | | | |
| 11V, 72A | 0.363 | 0.932 | 0.763 | 0.825 | 0.552 | 0.732 | | | |
| 14I 72G 117V | 0.312 | 0.928 | 0.547 | 0.622 | 0.337 | 0.085 | | | |
| 14V 18L 73V 117A 72A | 0.110 | 0.926 | 0.624 | 0.557 | 0.307 | 0.249 | 0.597 | 0.105 | |
| 115N, 72A | 0.164 | 0.918 | 0.726 | 0.777 | 0.711 | 0.558 | | | |
| 14L 18L 73L 117A 72A | 0.372 | 0.905 | 0.641 | 0.657 | 0.355 | 0.071 | 0.410 | 0.031 | |
| 115H, 72A | 0.155 | 0.891 | 0.697 | 0.741 | 0.675 | 0.514 | | | |
| 14Q 18S 73I 117W 72A | 0.487 | 0.878 | 0.894 | 0.784 | 0.809 | 0.559 | 0.834 | −0.044 | |
| 14L 72G 117V | 0.280 | 0.873 | 0.513 | 0.548 | 0.297 | 0.055 | | | |
| 14L 72G | 0.377 | 0.873 | 0.886 | 0.817 | 0.730 | 0.334 | | | |
| 73T, 72A | 0.157 | 0.872 | 0.765 | 0.768 | 0.778 | 0.621 | | | |
| 72G | 0.307 | 0.871 | 0.590 | 0.544 | 0.628 | 0.307 | | | |
| 104G, 72A | 0.162 | 0.866 | 0.687 | 0.665 | 0.691 | 0.650 | | | |
| 55L, 72A | 0.155 | 0.863 | 0.719 | 0.706 | 0.822 | 0.631 | | | |
| 102F, 72A | 0.218 | 0.858 | 0.588 | 0.657 | 0.554 | 0.369 | | | |
| 14L 72A | 0.241 | 0.838 | 0.867 | 0.766 | 0.446 | 0.534 | | | |
| 119S, 72A | 0.313 | 0.781 | 0.734 | 0.457 | 0.714 | 0.373 | | | |
| 82V, 72A | 0.263 | 0.757 | 0.456 | 0.519 | 0.461 | 0.345 | | | |
| 73L, 72A | 0.294 | 0.733 | 0.338 | 0.325 | 0.380 | 0.251 | | | |
| 11M, 72A | 0.325 | 0.697 | 0.651 | 0.576 | 0.463 | 0.543 | | | |
| 104F, 72A | 0.344 | 0.686 | 0.436 | 0.486 | 0.462 | 0.263 | | | |
| 68L, 72A | 0.207 | 0.658 | 0.520 | 0.530 | 0.547 | 0.524 | | | |
| 17I, 72A | 0.258 | 0.640 | 0.541 | 0.499 | 0.624 | 0.283 | | | |
| 17V, 72A | 0.305 | 0.639 | 0.586 | 0.549 | 0.558 | 0.308 | | | |
| 34E, 72A | 0.317 | 0.618 | 0.571 | 0.604 | 0.486 | 0.482 | | | |
| 18L 73V 72A | 0.364 | 0.615 | 0.126 | 0.069 | 0.142 | 0.083 | 0.107 | 0.359 | |
| 55Q, 72A | 0.225 | 0.600 | 0.492 | 0.474 | 0.536 | 0.428 | | | |
| 17M, 72A | 0.296 | 0.590 | 0.532 | 0.489 | 0.490 | 0.281 | | | |
| 55V, 72A | 0.169 | 0.581 | 0.477 | 0.432 | 0.468 | 0.374 | | | |
| 106M, 72A | 0.231 | 0.554 | 0.490 | 0.485 | 0.510 | 0.183 | | | |
| 117W, 72A | 0.294 | 0.551 | 0.461 | 0.446 | 0.388 | 0.339 | | | |
| 119A, 72A | 0.304 | 0.540 | 0.520 | 0.450 | 0.490 | 0.200 | | | |
| 21I, 72A | 0.241 | 0.531 | 0.412 | 0.365 | 0.310 | 0.162 | | | |
| 17L, 72A | 0.251 | 0.525 | 0.447 | 0.420 | 0.459 | 0.279 | | | |
| 11Y, 72A | 0.309 | 0.516 | 0.311 | 0.330 | 0.156 | 0.296 | | | |
| 23Y, 72A | 0.265 | 0.492 | 0.443 | 0.442 | 0.412 | 0.281 | | | |
| 55Y, 72A | 0.124 | 0.471 | 0.335 | 0.324 | 0.329 | 0.249 | | | |
| 78A, 72A | 0.239 | 0.433 | 0.429 | 0.372 | 0.350 | 0.190 | | | |
| 11W, 72A | 0.262 | 0.430 | 0.357 | 0.344 | 0.227 | 0.199 | | | |
| 104Y, 72A | 0.878 | 0.364 | 0.278 | 0.265 | 0.236 | 0.140 | | | |
| 38Y 18L 73V 72A | 0.386 | 1.172 | 0.133 | 0.034 | 0.235 | 0.067 | 0.023 | 0.824 | AA and SA |
| | | | | | | | | | specific |
| 18L 73V 72A | 0.455 | 0.475 | −0.028 | −0.092 | 0.030 | 0.010 | −0.027 | 0.427 | |
| 18L 55L 73I 72A | 0.474 | 0.443 | −0.044 | −0.129 | 0.021 | 0.007 | −0.030 | 0.423 | |
| 18L 73L 72A | 0.283 | 0.441 | 0.090 | 0.019 | 0.021 | −0.004 | 0.092 | 0.254 | |
| 73I 72A | 0.276 | 0.408 | 0.099 | 0.039 | 0.045 | 0.015 | 0.117 | 0.242 | |
| 18G 31F 73L 72A | 0.225 | 1.785 | 1.235 | 1.520 | 1.365 | 1.025 | 1.235 | 1.690 | |
| 72F | 0.520 | 0.401 | 0.592 | 0.612 | 0.571 | 0.456 | | | AA lowest |
| 72Y | 0.386 | 0.346 | 0.594 | 0.637 | 0.557 | 0.415 | | | |
| 74F, 72A | 0.28 | 0.44 | 1.04 | 0.67 | 0.51 | 0.93 | | | |
| 72T 73W 74G | 0.205 | 0.687 | 1.702 | 1.348 | 0.822 | 1.817 | 2.770 | 0.305 | SA and AA lowest |
| 38Y 74F 72A | 0.191 | 0.605 | 1.166 | 0.926 | 0.646 | 1.397 | 1.514 | 0.362 | |
| 73W 74I | 0.335 | 0.757 | 1.325 | 1.130 | 0.912 | 1.884 | 2.697 | 0.423 | |
| 73W 74T | 0.368 | 1.456 | 3.286 | 2.480 | 1.902 | 3.883 | 5.818 | 0.726 | |
| 60T 104T 115W 74F 72A | 0.136 | 0.873 | 2.017 | 1.609 | 1.149 | 1.920 | 2.511 | 0.633 | |
| 72V 73L 74W | 0.248 | 0.500 | 1.209 | 1.225 | 0.648 | 1.276 | 1.710 | 0.348 | |
| 72H 73P 74L | 0.933 | 4.597 | 6.352 | 5.429 | 3.919 | 5.260 | 7.165 | 1.474 | Large Response, Favors |
| | | | | | | | | | POA, LNA |
| 72G 73W 74E | 0.576 | 4.229 | 5.501 | 5.045 | 2.755 | 3.944 | 6.069 | 0.955 | |
| 72A 73W 74E | 0.503 | 4.093 | 5.422 | 4.686 | 3.277 | 4.937 | 6.615 | 1.289 | |
| 72T 73W 74S | 0.574 | 4.129 | 4.826 | 4.612 | 4.177 | 4.155 | 5.826 | 1.620 | |
| 72S 73W 74E | 0.520 | 3.751 | 4.799 | 4.079 | 3.212 | 4.210 | 6.109 | 1.285 | |
| 72A 73W 74S | 0.353 | 3.211 | 4.401 | 3.806 | 3.155 | 4.039 | 4.864 | 1.095 | |
| 72S 73W 74N | 0.639 | 3.770 | 4.220 | 3.977 | 3.490 | 3.333 | 4.867 | 1.255 | |
| 72S 73W 74S | 0.597 | 3.545 | 4.147 | 3.787 | 2.863 | 3.536 | 4.656 | 1.106 | |
| 73W 72A 74F | 0.567 | 3.378 | 3.875 | 3.718 | 3.255 | 3.264 | 5.256 | 1.485 | |
| 72T 73W 74E | 0.312 | 2.442 | 3.818 | 3.254 | 2.200 | 3.842 | 5.353 | 0.877 | |
| 72T 74V | 0.249 | 2.680 | 3.680 | 3.630 | 2.957 | 3.149 | 4.074 | 1.781 | |
| 72G 73W | 0.602 | 2.260 | 3.637 | 2.698 | 1.106 | 2.968 | 4.082 | 0.294 | |
| 72T 73V 74V | 0.215 | 3.237 | 3.618 | 3.502 | 3.166 | 2.987 | 4.015 | 1.918 | |
| 72T 73V 74L | 0.386 | 2.774 | 3.508 | 3.165 | 2.832 | 2.674 | 3.642 | 1.411 | |
| 72W 73T 74G | 0.500 | 2.808 | 3.394 | 3.099 | 2.514 | 2.934 | 4.105 | 1.040 | |
| 72S 73L 74A | 0.347 | 3.336 | 3.322 | 3.455 | 3.495 | 3.293 | 3.923 | 2.574 | |
| 72T 74I | 0.269 | 2.502 | 3.169 | 3.156 | 2.660 | 2.476 | 3.410 | 1.806 | |
| 72S 73V 74V | 0.356 | 2.712 | 3.088 | 3.085 | 2.556 | 2.091 | 3.322 | 1.058 | |
| 72T 73W 74L | 0.292 | 2.721 | 3.009 | 2.819 | 2.340 | 2.727 | 3.574 | 1.217 | |
| 72T 73Q 74A | 0.257 | 2.392 | 2.850 | 3.163 | 3.136 | 3.473 | 3.632 | 2.318 | |
| 72T 73V 74S | 0.241 | 2.540 | 2.579 | 2.611 | 2.670 | 2.629 | 2.909 | 2.195 | |
| 72S 73M 74A | 0.358 | 2.759 | 2.567 | 2.470 | 2.443 | 2.524 | 2.854 | 1.776 | |
| 72T 73V 74N | 0.280 | 2.484 | 2.362 | 2.407 | 2.491 | 2.250 | 2.816 | 1.942 | |
| 72S 73P 74I | 0.324 | 2.154 | 3.091 | 2.792 | 2.162 | 2.210 | 3.155 | 1.337 | Favors POA and LNA |
| 72A 73P 74V | 0.377 | 2.354 | 2.845 | 2.735 | 2.187 | 2.406 | 3.613 | 1.178 | |
| 18V 31A 73W 72A | 0.352 | 1.761 | 2.770 | 2.540 | 1.738 | 2.218 | 3.184 | 0.722 | |
| 72A 73W 74V | 0.393 | 1.768 | 2.752 | 2.338 | 1.574 | 2.525 | 3.856 | 0.602 | |
| 72T 73V 74A | 0.242 | 2.180 | 2.597 | 2.435 | 1.950 | 2.362 | 3.011 | 1.146 | |
| 72G 73F 74E | 0.381 | 0.910 | 2.415 | 1.467 | 0.585 | 0.981 | 2.519 | 0.239 | |
| 18I 31I 55M 73V 72A | 0.362 | 1.710 | 2.408 | 1.981 | 1.188 | 1.883 | 2.478 | 0.502 | |
| 72V 73W 74N | 0.207 | 1.191 | 2.384 | 1.726 | 1.146 | 2.229 | 3.507 | 0.468 | |
| 18I 31A 73F 72A | 0.330 | 1.314 | 2.318 | 1.732 | 0.950 | 1.515 | 2.730 | 0.404 | |
| 18I 31A 73F 72A | 0.330 | 1.296 | 2.203 | 1.652 | 0.995 | 1.372 | 2.522 | 0.406 | |
| 18I 31Y 73V 72A | 0.365 | 1.801 | 2.176 | 2.242 | 1.818 | 1.615 | 2.088 | 1.047 | |
| 72A 73W 74A | 0.268 | 0.892 | 2.093 | 1.466 | 0.933 | 1.942 | 3.016 | 0.330 | |
| 72V 73W 74S | 0.270 | 1.424 | 2.044 | 1.819 | 1.477 | 1.789 | 2.662 | 0.574 | |
| 18V 31A 73F 72A | 0.308 | 1.142 | 2.036 | 1.527 | 0.828 | 1.510 | 2.677 | 0.375 | |
| 60T 104T 115W 74F 72A | 0.136 | 0.873 | 2.017 | 1.609 | 1.149 | 1.920 | 2.511 | 0.633 | |
| 14Q 18L 31A 73W 117V 72A | 0.334 | 1.558 | 1.958 | 1.942 | 1.478 | 1.084 | 2.008 | 0.384 | |
| 18I 31V 73G 72A | 0.360 | 1.348 | 1.946 | 1.715 | 0.968 | 1.630 | 2.122 | 0.543 | |
| 18V 31A 55W 73W 72A | 0.344 | 1.130 | 1.887 | 1.555 | 0.913 | 1.505 | 2.042 | 0.392 | |
| 18I 31I 55M 73I 72A | 0.391 | 1.354 | 1.803 | 1.516 | 0.822 | 1.546 | 1.903 | 0.382 | |
| 72I 73W 74N | 0.229 | 0.791 | 1.794 | 1.194 | 0.779 | 2.091 | 3.236 | 0.329 | |
| 73F 72A | 0.299 | 1.051 | 1.775 | 1.171 | 0.630 | 1.118 | 2.381 | 0.399 | |
| 18I 31L 55L 72A | 0.318 | 1.285 | 1.736 | 1.442 | 0.759 | 1.523 | 2.011 | 0.310 | |
| 72S 73W 74G | 0.321 | 1.012 | 1.706 | 1.375 | 0.970 | 1.653 | 2.422 | 0.411 | |
| 73T 72A 74F | 0.300 | 0.939 | 1.661 | 1.389 | 1.161 | 1.632 | 2.450 | 0.519 | |
| 74T 72A 73F | 0.181 | 0.852 | 1.635 | 1.185 | 0.634 | 1.150 | 2.309 | 0.273 | |
| 55V 73F 72A | 0.328 | 0.864 | 1.566 | 1.223 | 0.661 | 1.082 | 2.264 | 0.285 | |
| 74E 72A 73F | 0.259 | 0.703 | 1.558 | 1.093 | 0.575 | 1.026 | 2.395 | 0.251 | |
| 73P 72A 74F | 0.265 | 1.078 | 1.518 | 0.838 | 0.868 | 1.278 | 1.776 | 0.494 | |
| 18V 31F 73M 72A | 0.314 | 1.014 | 1.511 | 1.296 | 0.794 | 1.395 | 1.538 | 0.383 | |
| 74S 72A 73F | 0.230 | 0.893 | 1.433 | 1.035 | 0.630 | 1.158 | 2.413 | 0.337 | |
| 38I 74F 72A | 0.276 | 0.787 | 1.395 | 1.162 | 0.764 | 1.343 | 2.112 | 0.503 | |
| 72G 73F 74N | 0.513 | 0.617 | 1.367 | 0.454 | −0.032 | 0.456 | 1.499 | −0.043 | |
| 73W 74I | 0.335 | 0.757 | 1.325 | 1.130 | 0.912 | 1.884 | 2.697 | 0.423 | |
| 72V 73L 74W | 0.248 | 0.500 | 1.209 | 1.225 | 0.648 | 1.276 | 1.710 | 0.348 | |
| 18V 31F 55L 72A | 0.303 | 0.921 | 1.180 | 0.996 | 0.649 | 1.035 | 1.353 | 0.358 | |
| 18I 31V 55V 73V 72A | 0.362 | 0.632 | 1.179 | 0.853 | 0.448 | 0.974 | 1.236 | 0.233 | |
| 74Q 72A 73F | 0.251 | 0.465 | 1.025 | 0.711 | 0.390 | 0.649 | 1.479 | 0.177 | |
| 72E 73V 74A | 0.296 | 0.478 | 0.784 | 0.735 | 0.524 | 0.736 | 1.093 | 0.234 | |
| 72G 73F 74E | 0.381 | 0.910 | 2.415 | 1.467 | 0.585 | 0.981 | 2.519 | 0.239 | |
| 18I 31A 73F 72A | 0.330 | 1.296 | 2.203 | 1.652 | 0.995 | 1.372 | 2.522 | 0.406 | |
| 18I 31L 55L 72A | 0.318 | 1.285 | 1.736 | 1.442 | 0.759 | 1.523 | 2.011 | 0.310 | |
| 72A 73P 74E | 0.199 | 1.975 | 1.863 | 1.741 | 1.802 | 1.732 | 2.087 | 1.512 | |
| 18I 31V 55V 73V 72A | 0.362 | 0.632 | 1.179 | 0.853 | 0.448 | 0.974 | 1.236 | 0.233 | |
| 60T 104T 115W 74F 72A | 0.136 | 0.873 | 2.017 | 1.609 | 1.149 | 1.920 | 2.511 | 0.633 | |
| 18V 31A 55W 73W 72A | 0.344 | 1.130 | 1.887 | 1.555 | 0.913 | 1.505 | 2.042 | 0.392 | |
| 18I 31I 55M 73I 72A | 0.391 | 1.354 | 1.803 | 1.516 | 0.822 | 1.546 | 1.903 | 0.382 | |
| 18I 31I 55M 73V 72A | 0.362 | 1.710 | 2.408 | 1.981 | 1.188 | 1.883 | 2.478 | 0.502 | |
| 73P 72A 74F | 0.265 | 1.078 | 1.518 | 0.838 | 0.868 | 1.278 | 1.776 | 0.494 | |
| 18V 31A 73W 72A | 0.352 | 1.761 | 2.770 | 2.540 | 1.738 | 2.218 | 3.184 | 0.722 | |
| 14Q 18L 31A 73W 117V 72A | 0.334 | 1.558 | 1.958 | 1.942 | 1.478 | 1.084 | 2.008 | 0.384 | |
| 18I 31M 55M 72A | 0.352 | 1.010 | 1.358 | 1.134 | 0.601 | 1.099 | 1.353 | 0.296 | |
| 18I 31V 73G 72A | 0.360 | 1.348 | 1.946 | 1.715 | 0.968 | 1.630 | 2.122 | 0.543 | |
| 18V 31F 55L 72A | 0.303 | 0.921 | 1.180 | 0.996 | 0.649 | 1.035 | 1.353 | 0.358 | |
| 18V 31F 73M 72A | 0.314 | 1.014 | 1.511 | 1.296 | 0.794 | 1.395 | 1.538 | 0.383 | |
| 18V 31A 55I 72A | 0.345 | 0.989 | 1.580 | 1.334 | 0.791 | 1.280 | 1.563 | 0.420 | |
| 72T 73V 74A | 0.242 | 2.180 | 2.597 | 2.435 | 1.950 | 2.362 | 3.011 | 1.146 | |
| 72S 73P 74I | 0.324 | 2.154 | 3.091 | 2.792 | 2.162 | 2.210 | 3.155 | 1.337 | |
| 72T 73I 74A | 0.293 | 2.148 | 1.944 | 1.795 | 1.721 | 1.687 | 2.320 | 1.359 | |
| 72S 74A | 0.208 | 1.648 | 2.002 | 2.037 | 1.575 | 1.615 | 2.034 | 1.114 | |
| 72S 73V 74A | 0.340 | 2.127 | 2.330 | 2.397 | 2.295 | 2.601 | 2.727 | 1.572 | |
| 72T 73G 74I | 0.311 | 2.226 | 2.317 | 2.577 | 2.414 | 2.177 | 2.771 | 1.755 | |
| 72S 73Q 74A | 0.295 | 2.290 | 2.077 | 2.140 | 1.875 | 1.896 | 2.293 | 1.311 | |
| 18I 31F 72A | 0.314 | 2.043 | 3.027 | 2.707 | 1.761 | 2.037 | 2.659 | 0.950 | Favors LNA |
| 18I 31Y 72A | 0.345 | 2.205 | 2.813 | 2.790 | 2.096 | 1.762 | 2.639 | 0.994 | |
| 18I 31Y 73G 72A | 0.298 | 1.796 | 2.706 | 2.559 | 1.632 | 1.886 | 2.637 | 0.860 | |
| 72T 74S | 0.251 | 2.035 | 2.621 | 2.469 | 2.252 | 2.209 | 2.471 | 1.779 | |
| 14R 18L 31S 73F 117E 72A | 0.524 | 1.241 | 2.296 | 1.947 | 1.146 | 0.693 | 1.290 | 0.378 | |
| 14R 18L 73L 117D 72A | 0.647 | 1.029 | 1.790 | 1.766 | 0.847 | 0.294 | 0.801 | 0.213 | |
| 72A 74N | 0.162 | 1.614 | 1.701 | 1.681 | 1.629 | 1.308 | 1.536 | 1.299 | |
| 14L 18L 31S 73W 117V 72A | 0.264 | 1.458 | 1.631 | 1.569 | 1.208 | 0.692 | 1.356 | 0.329 | |
| 18V 31A 55I 72A | 0.345 | 0.989 | 1.580 | 1.334 | 0.791 | 1.280 | 1.563 | 0.420 | |
| 18V 31V 73I 72A | 0.329 | 0.863 | 1.531 | 1.151 | 0.670 | 1.302 | 1.334 | 0.311 | |
| 18I 31L 73G 72A | 0.367 | 0.981 | 1.525 | 1.146 | 0.595 | 1.137 | 1.377 | 0.267 | |
| 14L 18L 73W 117S 72A | 0.562 | 1.010 | 1.499 | 1.385 | 0.374 | 0.259 | 0.750 | −0.005 | |
| 38W 106H 117A 72A | 0.414 | 1.326 | 1.397 | 1.248 | 0.831 | 0.426 | 1.098 | 0.071 | |
| 18I 31L 72A | 0.310 | 1.054 | 1.384 | 1.198 | 0.719 | 1.182 | 1.187 | 0.305 | |
| 18I 31M 55M 72A | 0.352 | 1.010 | 1.358 | 1.134 | 0.601 | 1.099 | 1.353 | 0.296 | |
| 18I 55G 73M 72A | 0.303 | 0.895 | 1.284 | 1.127 | 0.574 | 1.137 | 1.201 | 0.360 | |
| 18V 31Y 55I 73G 72A | 0.383 | 1.007 | 1.228 | 1.220 | 0.994 | 0.780 | 1.106 | −0.094 | |
| 14Q 18S 73I 117W 72A | 0.487 | 0.878 | 0.894 | 0.784 | 0.809 | 0.559 | 0.834 | −0.044 | |
| 119M, 72A | 0.343 | 1.087 | 2.280 | 1.184 | 1.191 | 0.722 | | | Favors LNA |
| 78F, 72A | 0.219 | 2.001 | 2.033 | 1.955 | 1.912 | 1.416 | | | |
| 14M 72M 117A | 0.305 | 1.649 | 2.031 | 1.956 | 1.054 | 0.870 | | | |
| 38Q, 72A | 0.204 | 1.976 | 1.986 | 1.936 | 1.842 | 1.551 | | | |
| 73F, 72A | 0.330 | 1.208 | 1.861 | 1.234 | 0.728 | 1.128 | | | |
| 38T, 72A | 0.297 | 1.598 | 1.639 | 1.577 | 1.547 | 1.022 | | | |
| 17W, 72A | 0.175 | 1.421 | 1.611 | 1.416 | 1.241 | 1.067 | | | |
| 62P, 72A | 0.216 | 1.548 | 1.581 | 1.572 | 1.448 | 1.247 | | | |
| 31Q, 72A | 0.376 | 1.024 | 1.494 | 1.465 | 1.313 | 1.039 | | | |
| 38S, 72A | 0.311 | 1.175 | 1.295 | 1.286 | 1.235 | 0.724 | | | |
| 14W 72M 117A | 0.322 | 0.945 | 1.286 | 1.009 | 0.706 | 0.515 | | | |
| 36V, 72A | 0.330 | 1.026 | 1.082 | 1.062 | 0.869 | 0.713 | | | |
| 74F, 72A | 0.28 | 0.44 | 1.04 | 0.67 | 0.51 | 0.93 | | | |
| 36I, 72A | 0.250 | 0.812 | 1.004 | 0.884 | 0.773 | 0.815 | | | |
| 23W, 72A | 0.449 | 0.618 | 0.956 | 0.794 | 0.448 | 0.492 | | | |
| 17Y, 72A | 0.647 | 0.729 | 0.806 | 0.712 | 0.732 | 0.500 | | | |
| 31V, 72A | 0.613 | 0.395 | 0.776 | 0.384 | 0.081 | 0.206 | | | |
| 14M, 72A | 0.326 | 0.619 | 0.684 | 0.685 | 0.412 | 0.452 | | | |
| 117D, 72A | 0.389 | 0.352 | 0.407 | 0.380 | 0.294 | 0.142 | | | |
| 31I, 72A | 0.680 | 0.303 | 0.413 | 0.168 | −0.213 | 0.091 | | | Favors LNA largest OA neg |
| 104N, 72A | 0.396 | 1.018 | 0.391 | 0.385 | 0.408 | 0.406 | | | LNA lowest |
| 14L 18L 31Y 73L 117A 72A | 0.581 | 2.987 | 2.818 | 2.948 | 1.908 | 1.444 | 2.317 | 0.762 | Favors PUFA, |
| | | | | | | | | | POA |
| 18I 31F 73V 72A | 0.355 | 2.649 | 2.738 | 3.096 | 2.257 | 1.893 | 2.665 | 1.294 | |
| 104T 115A 74F 72A | 0.287 | 2.635 | 2.206 | 2.231 | 1.621 | 0.720 | 1.661 | 0.877 | |
| 18I 31Y 73I 72A | 0.340 | 2.487 | 2.865 | 3.031 | 2.322 | 1.825 | 2.620 | 1.310 | |
| 18L 73F 117G 72A | 0.400 | 2.445 | 1.913 | 2.283 | 1.908 | 1.039 | 1.731 | 0.663 | |
| 18V 31Y 73V 72A | 0.343 | 2.400 | 2.605 | 2.780 | 2.349 | 1.534 | 2.376 | 1.330 | |
| 18V 31I 55L 73V 72A | 0.316 | 2.273 | 2.433 | 2.680 | 2.254 | 1.253 | 2.285 | 1.233 | |
| 14A 18Y 31W 73G 117V 72A | 0.261 | 0.365 | 0.940 | 1.018 | 0.518 | 0.421 | 0.883 | 0.158 | Favors LNA, LA |
| 31R, 72A | 0.182 | 0.250 | 0.437 | 0.405 | 0.227 | 0.342 | | | |
| 31E, 72A | 0.308 | 0.238 | 0.522 | 0.430 | 0.211 | 0.223 | | | |
| 74L, 72A | 0.172 | 0.222 | 0.404 | 0.305 | 0.188 | 0.361 | | | |
| 31T, 72A | 0.389 | 0.143 | 0.510 | 0.215 | −0.024 | 0.145 | | | |
| 18V 31Y 73I 72A | 0.359 | 1.946 | 2.537 | 2.619 | 2.007 | 1.784 | 2.528 | 1.139 | Favors LA, some LNA about |
| | | | | | | | | | same |
| 78F 102I 72A | 0.297 | 2.093 | 2.322 | 2.493 | 2.506 | 1.434 | 2.022 | 1.877 | |
| 38W, 72A | 0.231 | 1.350 | 1.336 | 2.413 | 1.583 | 1.552 | | | |
| 62W, 72A | 0.211 | 2.206 | 2.120 | 2.339 | 2.155 | 1.450 | | | |
| 18I 31Y 73V 72A | 0.365 | 1.801 | 2.176 | 2.242 | 1.818 | 1.615 | 2.088 | 1.047 | |
| 14I 18L 31W 73V 117G 72A | 0.470 | 2.037 | 1.960 | 2.047 | 1.756 | 1.236 | 1.770 | 0.606 | |
| 72S 74A | 0.208 | 1.648 | 2.002 | 2.037 | 1.575 | 1.615 | 2.034 | 1.114 | |
| 14M 18L 31W 73I 117G 72A | 0.580 | 2.032 | 1.869 | 2.034 | 1.712 | 0.998 | 1.569 | 0.487 | |
| 14L 31W 117V 72A | 0.259 | 1.972 | 1.894 | 2.027 | 1.580 | 1.304 | 1.768 | 0.742 | |
| 14Q 18L 31A 73W 117V 72A | 0.334 | 1.558 | 1.958 | 1.942 | 1.478 | 1.084 | 2.008 | 0.384 | |
| 49I, 72A | 0.362 | 1.796 | 1.820 | 1.839 | 1.804 | 1.162 | | | |
| 70Q, 72A | 0.307 | 1.546 | 1.622 | 1.819 | 1.604 | 0.868 | | | |
| 14L 18L 31W 73W 117L 72A | 0.201 | 1.772 | 1.640 | 1.782 | 1.085 | 0.970 | 2.059 | 0.294 | |
| 38M 104S 115A 74F 72A | 0.325 | 1.690 | 1.615 | 1.778 | 1.155 | 0.498 | 1.172 | 0.356 | |
| 14R 18L 73L 117D 72A | 0.647 | 1.029 | 1.790 | 1.766 | 0.847 | 0.294 | 0.801 | 0.213 | |
| 31F, 72A | 0.492 | 1.210 | 1.647 | 1.759 | 1.331 | 1.438 | | | |
| 23V, 72A | 0.167 | 1.464 | 1.351 | 1.731 | 1.561 | 1.475 | | | |
| 60I, 72A | 0.202 | 1.539 | 1.632 | 1.730 | 1.723 | 1.520 | | | |
| 11Q, 72A | 0.262 | 1.525 | 1.582 | 1.715 | 1.716 | 1.450 | | | |
| 104S, 72A | 0.242 | 1.638 | 1.538 | 1.715 | 1.663 | 1.570 | | | |
| 73Y, 72A | 0.282 | 1.529 | 1.603 | 1.711 | 1.537 | 0.982 | | | |
| 72A 74N | 0.162 | 1.614 | 1.701 | 1.681 | 1.629 | 1.308 | 1.536 | 1.299 | |
| 14I 18V 31W 73I 117V 72A | 0.287 | 1.553 | 1.495 | 1.668 | 0.887 | 0.652 | 1.411 | 0.283 | |
| 62D, 72A | 0.230 | 1.485 | 1.576 | 1.598 | 1.574 | 1.385 | | | |
| 18L, 72A | 0.265 | 1.490 | 1.544 | 1.596 | 1.551 | 1.090 | | | |
| 14L 18L 31S 73W 117V 72A | 0.264 | 1.458 | 1.631 | 1.569 | 1.208 | 0.692 | 1.356 | 0.329 | |
| 21D, 72A | 0.359 | 1.301 | 1.478 | 1.562 | 1.345 | 0.707 | | | |
| 117S, 72A | 0.357 | 1.467 | 1.133 | 1.533 | 1.258 | 0.593 | | | |
| 31Q, 72A | 0.376 | 1.024 | 1.494 | 1.465 | 1.313 | 1.039 | | | |
| 14L 18L 73W 117S 72A | 0.562 | 1.010 | 1.499 | 1.385 | 0.374 | 0.259 | 0.750 | −0.005 | |
| 117Q, 72A | 0.368 | 1.214 | 1.236 | 1.374 | 1.134 | 0.631 | | | |
| 49M, 72A | 0.347 | 1.363 | 1.369 | 1.370 | 1.296 | 0.939 | | | |
| 38Q 62I 106I 117A 72A | 0.215 | 1.656 | 0.930 | 1.288 | 1.167 | 0.550 | 0.473 | 0.663 | |
| 60L, 72A | 0.236 | 1.075 | 1.260 | 1.285 | 1.272 | 1.095 | | | |
| 18V 31Y 55I 73G 72A | 0.383 | 1.007 | 1.228 | 1.220 | 0.994 | 0.780 | 1.106 | −0.094 | |
| 47M, 72A | 0.225 | 1.138 | 1.135 | 1.183 | 1.168 | 0.966 | | | |
| 31W, 72A | 0.462 | 0.993 | 1.031 | 1.147 | 0.868 | 0.618 | | | |
| 119L, 72A | 0.349 | 1.021 | 1.053 | 1.134 | 0.778 | 0.661 | | | |
| 74E 72A 73F | 0.259 | 0.703 | 1.558 | 1.093 | 0.575 | 1.026 | 2.395 | 0.251 | |
| 14L 38I 72A | 0.267 | 1.046 | 1.177 | 1.090 | 0.653 | 0.712 | | | |
| 40M, 72A | 0.195 | 0.993 | 1.021 | 1.086 | 0.990 | 0.691 | | | |
| 14W 18L 117S 72A | 0.228 | 0.976 | 0.835 | 1.063 | 0.904 | 0.377 | 0.811 | 0.297 | |
| 36V, 72A | 0.330 | 1.026 | 1.082 | 1.062 | 0.869 | 0.713 | | | |
| 40V, 72A | 0.164 | 1.013 | 1.059 | 1.057 | 1.002 | 0.867 | | | |
| 117H, 72A | 0.311 | 0.913 | 0.924 | 1.056 | 1.012 | 0.659 | | | |
| 117A, 72A | 0.270 | 1.332 | 0.631 | 1.045 | 0.947 | 0.331 | | | |
| 126K, 72A | 0.455 | 0.625 | 1.017 | 1.039 | 0.631 | 0.356 | | | |
| 38G, 72A | 0.301 | 0.996 | 0.992 | 1.003 | 0.945 | 0.447 | | | |
| 60A, 72A | 0.227 | 0.787 | 0.938 | 0.977 | 0.936 | 0.742 | | | |
| 18K, 72A | 0.359 | 0.800 | 0.925 | 0.967 | 0.803 | 0.544 | | | |
| 70H, 72A | 0.374 | 0.751 | 0.886 | 0.929 | 0.836 | 0.567 | | | |
| 36Q, 72A | 0.247 | 0.837 | 0.880 | 0.927 | 0.792 | 0.777 | | | |
| 117V, 72A | 0.153 | 0.805 | 0.767 | 0.924 | 0.774 | 0.498 | | | |
| 117T, 72A | 0.197 | 0.791 | 0.775 | 0.917 | 0.813 | 0.567 | | | |
| 60W, 72A | 0.185 | 0.670 | 0.739 | 0.837 | 0.763 | 0.595 | | | |
| 14V, 72A | 0.317 | 0.756 | 0.868 | 0.836 | 0.513 | 0.430 | | | |
| 106F, 72A | 0.294 | 0.754 | 0.706 | 0.812 | 0.815 | 0.369 | | | |
| 70S, 72A | 0.422 | 0.625 | 0.703 | 0.794 | 0.727 | 0.416 | | | |
| 14I, 72A | 0.391 | 0.572 | 0.899 | 0.792 | 0.572 | 0.586 | | | |
| 117I, 72A | 0.133 | 0.674 | 0.666 | 0.789 | 0.676 | 0.408 | | | |
| 117E, 72A | 0.196 | 0.656 | 0.638 | 0.753 | 0.659 | 0.402 | | | |
| 70W, 72A | 0.388 | 0.349 | 0.385 | 0.451 | 0.381 | 0.212 | | | |
| 18L 73V 72A | 0.364 | 0.615 | 0.126 | 0.069 | 0.142 | 0.083 | 0.107 | 0.359 | LA low |
| 73V, 72A | 0.300 | 0.976 | 0.496 | 0.396 | 0.603 | 0.475 | | | |
| 119S, 72A | 0.313 | 0.781 | 0.734 | 0.457 | 0.714 | 0.373 | | | LA & PA low |
| 72V 73L 74W | 0.248 | 0.500 | 1.209 | 1.225 | 0.648 | 1.276 | 1.710 | 0.348 | LA, POA high |
| 14L 18L 31L 73G 117V 72A | 0.440 | 1.180 | 1.139 | 1.170 | 0.031 | 0.377 | 0.180 | 0.170 | PUFA high |
| 78F 102I 72A | 0.297 | 2.093 | 2.322 | 2.493 | 2.506 | 1.434 | 2.022 | 1.877 | Favors OA |
| 102I, 72A | 0.217 | 1.461 | 1.763 | 1.902 | 1.929 | 1.169 | | | |
| 60F, 72A | 0.247 | 1.792 | 1.762 | 1.684 | 1.896 | 1.526 | | | |
| 91Y, 72A | 0.307 | 1.273 | 1.626 | 1.415 | 1.706 | 0.870 | | | |
| 38V 62V 117A 72A | 0.325 | 0.984 | 1.153 | 1.002 | 1.668 | 0.478 | 0.668 | 0.495 | |
| 91C, 72A | 0.334 | 1.201 | 1.420 | 1.371 | 1.470 | 0.794 | | | |
| 102L, 72A | 0.154 | 1.103 | 1.284 | 1.267 | 1.366 | 1.012 | | | |
| 34W, 72A | 0.187 | 1.060 | 1.130 | 1.110 | 1.210 | 0.920 | | | |
| 119I, 72A | 0.303 | 0.920 | 0.940 | 0.960 | 1.050 | 0.720 | | | |
| 117H, 72A | 0.311 | 0.913 | 0.924 | 1.056 | 1.012 | 0.659 | | | |
| 38H, 72A | 0.204 | 0.998 | 0.864 | 0.905 | 0.955 | 0.624 | | | |
| 18G 31W 73V 72A | 0.232 | 0.774 | 0.705 | 0.855 | 0.942 | 0.463 | 0.770 | 0.489 | |
| 102Y, 72A | 0.719 | 0.718 | 0.732 | 0.818 | 0.860 | 0.387 | | | |
| 72N | 0.310 | 0.652 | 0.702 | 0.770 | 0.831 | 0.441 | | | |
| 55L, 72A | 0.155 | 0.863 | 0.719 | 0.706 | 0.822 | 0.631 | | | |
| 106F, 72A | 0.294 | 0.754 | 0.706 | 0.812 | 0.815 | 0.369 | | | |
| 119V, 72A | 0.344 | 0.750 | 0.710 | 0.720 | 0.780 | 0.480 | | | |
| 93L, 72A | 0.312 | 0.680 | 0.680 | 0.670 | 0.770 | 0.560 | | | |
| 106H, 72A | 0.279 | 0.636 | 0.633 | 0.669 | 0.732 | 0.258 | | | |
| 106I, 72A | 0.291 | 0.481 | 0.624 | 0.563 | 0.661 | 0.223 | | | |
| 106V, 72A | 0.261 | 0.398 | 0.592 | 0.574 | 0.657 | 0.191 | | | |
| 60K, 72A | 0.465 | 0.461 | 0.582 | 0.619 | 0.626 | 0.310 | | | |
| 17I, 72A | 0.258 | 0.640 | 0.541 | 0.499 | 0.624 | 0.283 | | | |
| 106A, 72A | 0.249 | 0.389 | 0.518 | 0.515 | 0.596 | 0.164 | | | |
| 106N, 72A | 0.238 | 0.384 | 0.445 | 0.451 | 0.496 | 0.152 | | | |
| 106S, 72A | 0.258 | 0.321 | 0.403 | 0.355 | 0.478 | 0.149 | | | |
| 106T, 72A | 0.228 | 0.250 | 0.367 | 0.246 | 0.434 | 0.118 | | | |
| 106L, 72A | 0.299 | 0.383 | 0.364 | 0.382 | 0.431 | 0.183 | | | |
| 38A 106V 117A 72A | 0.418 | 1.492 | 0.541 | 0.229 | 0.368 | 0.033 | 0.121 | 0.065 | OA>>PA |
| 14L 18L 73L 117A 72A | 0.372 | 0.905 | 0.641 | 0.657 | 0.355 | 0.071 | 0.410 | 0.031 | |
| 38Y 62W 117A 72A | 0.554 | 2.353 | 1.237 | 1.848 | 1.896 | 0.316 | 0.272 | 0.854 | |
| 38W 106H 117A 72A | 0.414 | 1.326 | 1.397 | 1.248 | 0.831 | 0.426 | 1.098 | 0.071 | |
| 72G 73F 74N | 0.513 | 0.617 | 1.367 | 0.454 | −0.032 | 0.456 | 1.499 | −0.043 | Miscellaneous phenotypes |
| 14L 18L 31L 73G 117V 72A | 0.440 | 1.180 | 1.139 | 1.170 | 0.031 | 0.377 | 0.180 | 0.170 | |
| 31V, 72A | 0.613 | 0.395 | 0.776 | 0.384 | 0.081 | 0.206 | | | |
| 11Y, 72A | 0.309 | 0.516 | 0.311 | 0.330 | 0.156 | 0.296 | | | |
| 14I 38I 72V | 0.205 | 0.715 | 0.926 | 0.862 | 0.398 | 0.844 | | | |
| 14I 72A | 0.345 | 0.825 | 0.997 | 0.944 | 0.478 | 0.762 | | | |
| 73F, 72A | 0.330 | 1.208 | 1.861 | 1.234 | 0.728 | 1.128 | | | |
| 11I, 72A | 0.382 | 0.584 | 0.507 | 0.546 | 0.387 | 1.186 | | | |
| 14L 72A | 0.241 | 0.838 | 0.867 | 0.766 | 0.446 | 0.534 | | | |
| 14L 38M 72A 117F | 0.213 | 0.861 | 0.921 | 0.830 | 0.458 | 0.472 | | | |
| 73F 72A | 0.299 | 1.051 | 1.775 | 1.171 | 0.630 | 1.118 | 2.381 | 0.399 | |
| 72H 73Y 74G | 0.205 | 0.600 | 0.591 | 0.472 | 0.140 | 0.486 | 0.986 | 0.052 | |
| 31I, 72A | 0.680 | 0.303 | 0.413 | 0.168 | −0.213 | 0.091 | | | OA neg |
| 18V 31Y 55I 73G 72A | 0.383 | 1.007 | 1.228 | 1.220 | 0.994 | 0.780 | 1.106 | −0.094 | SA very low to |
| | | | | | | | | | zero |
| 14Q 18S 73I 117W 72A | 0.487 | 0.878 | 0.894 | 0.784 | 0.809 | 0.559 | 0.834 | −0.044 | |
| 14L 18L 73W 117S 72A | 0.562 | 1.010 | 1.499 | 1.385 | 0.374 | 0.259 | 0.750 | −0.005 | |
| 38W 106H 117A 72A | 0.414 | 1.326 | 1.397 | 1.248 | 0.831 | 0.426 | 1.098 | 0.071 | low SA somewhat low PA |
| 18V 31Y 55I 73G 72A | 0.383 | 1.007 | 1.228 | 1.220 | 0.994 | 0.780 | 1.106 | −0.094 | |
| 72H 73Y 74G | 0.205 | 0.600 | 0.591 | 0.472 | 0.140 | 0.486 | 0.986 | 0.052 | |
| 72G 73F 74N | 0.513 | 0.617 | 1.367 | 0.454 | −0.032 | 0.456 | 1.499 | −0.043 | low OA and SA, AA and |
| | | | | | | | | | POA high |
| 73W 74T | 0.368 | 1.456 | 3.286 | 2.480 | 1.902 | 3.883 | 5.818 | 0.726 | Favors POA and PA |
| 72T 73Q 74S | 0.283 | 2.166 | 2.419 | 2.713 | 2.676 | 2.646 | 3.110 | 1.792 | |
| 72S 73V 74A | 0.340 | 2.127 | 2.330 | 2.397 | 2.295 | 2.601 | 2.727 | 1.572 | |
| 72G 74F | 0.467 | 1.669 | 1.970 | 2.116 | 1.490 | 2.304 | 2.996 | 0.669 | |
| 72I 73W 74N | 0.229 | 0.791 | 1.794 | 1.194 | 0.779 | 2.091 | 3.236 | 0.329 | |
| 73W 74I | 0.335 | 0.757 | 1.325 | 1.130 | 0.912 | 1.884 | 2.697 | 0.423 | |
| 72T 73W 74G | 0.205 | 0.687 | 1.702 | 1.348 | 0.822 | 1.817 | 2.770 | 0.305 | |
| 73N 72A 74F | 0.448 | 0.870 | 1.299 | 0.976 | 0.775 | 1.468 | 2.256 | 0.271 | |
| 38Y 74F 72A | 0.191 | 0.605 | 1.166 | 0.926 | 0.646 | 1.397 | 1.514 | 0.362 | |
| 72V 73L 74W | 0.248 | 0.500 | 1.209 | 1.225 | 0.648 | 1.276 | 1.710 | 0.348 | |
| 11I, 72A | 0.382 | 0.584 | 0.507 | 0.546 | 0.387 | 1.186 | | | |
| 18I 31L 73L 72A | 0.380 | 0.626 | 0.857 | 0.682 | 0.549 | 1.075 | 0.781 | 0.390 | |
| 72Q | 0.665 | 0.770 | 0.735 | 0.762 | 0.777 | 0.989 | | | |
| 14L 18L 73L 117A 72A | 0.372 | 0.905 | 0.641 | 0.657 | 0.355 | 0.071 | 0.410 | 0.031 | PA & SA very low |
| 38A 106V 117A 72A | 0.418 | 1.492 | 0.541 | 0.229 | 0.368 | 0.033 | 0.121 | 0.065 | AA highest |
| 14I 72G 117V | 0.312 | 0.928 | 0.547 | 0.622 | 0.337 | 0.085 | | | AA highest |
| 14L 72G 117V | 0.280 | 0.873 | 0.513 | 0.548 | 0.297 | 0.055 | | | AA highest |
| 14L 18S 31L 73V 117A 72A | 0.486 | 0.951 | 0.673 | 0.534 | 0.020 | 0.028 | −0.003 | 0.007 | AA highest |
| 106D, 72A | 0.240 | 0.502 | 0.591 | 0.584 | 0.423 | 0.147 | | | |
| 31I, 72A | 0.680 | 0.303 | 0.413 | 0.168 | −0.213 | 0.091 | | | |
| 55Y, 72A | 0.124 | 0.471 | 0.335 | 0.324 | 0.329 | 0.249 | | | PA & SA low |
| 14L 72G 117I | 0.246 | 0.787 | 0.812 | 0.764 | 0.395 | 0.231 | | | |
| 82F, 72A | 0.139 | 1.029 | 0.334 | 0.422 | 0.403 | 0.223 | | | |
| 106I, 72A | 0.291 | 0.481 | 0.624 | 0.563 | 0.661 | 0.223 | | | |
| 70W, 72A | 0.388 | 0.349 | 0.385 | 0.451 | 0.381 | 0.212 | | | |
| 119A, 72A | 0.304 | 0.540 | 0.520 | 0.450 | 0.490 | 0.200 | | | |
| 11W, 72A | 0.262 | 0.430 | 0.357 | 0.344 | 0.227 | 0.199 | | | |
| 106V, 72A | 0.261 | 0.398 | 0.592 | 0.574 | 0.657 | 0.191 | | | |
| 78A, 72A | 0.239 | 0.433 | 0.429 | 0.372 | 0.350 | 0.190 | | | |
| 106M, 72A | 0.231 | 0.554 | 0.490 | 0.485 | 0.510 | 0.183 | | | |
| 106L, 72A | 0.299 | 0.383 | 0.364 | 0.382 | 0.431 | 0.183 | | | |
| 106A, 72A | 0.249 | 0.389 | 0.518 | 0.515 | 0.596 | 0.164 | | | |
| 21I, 72A | 0.241 | 0.531 | 0.412 | 0.365 | 0.310 | 0.162 | | | |
| 49D, 72A | 0.231 | 0.308 | 0.296 | 0.324 | 0.316 | 0.161 | | | |
| 106N, 72A | 0.238 | 0.384 | 0.445 | 0.451 | 0.496 | 0.152 | | | |
| 106S, 72A | 0.258 | 0.321 | 0.403 | 0.355 | 0.478 | 0.149 | | | |
| 117D, 72A | 0.389 | 0.352 | 0.407 | 0.380 | 0.294 | 0.142 | | | |
| 104Y, 72A | 0.878 | 0.364 | 0.278 | 0.265 | 0.236 | 0.140 | | | |
| 14L 18I 31L 73F 117A 72A | 0.255 | 1.297 | 0.639 | 0.707 | 0.300 | 0.121 | 0.235 | 0.077 | |
| 106T, 72A | 0.228 | 0.250 | 0.367 | 0.246 | 0.434 | 0.118 | | | |
| 18L 73V 72A | 0.364 | 0.615 | 0.126 | 0.069 | 0.142 | 0.083 | 0.107 | 0.359 | |
| 73W 74T | 0.368 | 1.456 | 3.286 | 2.480 | 1.902 | 3.883 | 5.818 | 0.726 | Favors POA |
| 72A 73W 74V | 0.393 | 1.768 | 2.752 | 2.338 | 1.574 | 2.525 | 3.856 | 0.602 | |
| 72A 73P 74V | 0.377 | 2.354 | 2.845 | 2.735 | 2.187 | 2.406 | 3.613 | 1.178 | |
| 72V 73W 74N | 0.207 | 1.191 | 2.384 | 1.726 | 1.146 | 2.229 | 3.507 | 0.468 | |
| 72I 73W 74N | 0.229 | 0.791 | 1.794 | 1.194 | 0.779 | 2.091 | 3.236 | 0.329 | |
| 72T 73Q 74S | 0.283 | 2.166 | 2.419 | 2.713 | 2.676 | 2.646 | 3.110 | 1.792 | |
| 72A 73W 74A | 0.268 | 0.892 | 2.093 | 1.466 | 0.933 | 1.942 | 3.016 | 0.330 | |
| 72G 74F | 0.467 | 1.669 | 1.970 | 2.116 | 1.490 | 2.304 | 2.996 | 0.669 | |
| 72T 73W 74G | 0.205 | 0.687 | 1.702 | 1.348 | 0.822 | 1.817 | 2.770 | 0.305 | |
| 18I 31A 73F 72A | 0.330 | 1.314 | 2.318 | 1.732 | 0.950 | 1.515 | 2.730 | 0.404 | |
| 73W 74I | 0.335 | 0.757 | 1.325 | 1.130 | 0.912 | 1.884 | 2.697 | 0.423 | |
| 18V 31A 73F 72A | 0.308 | 1.142 | 2.036 | 1.527 | 0.828 | 1.510 | 2.677 | 0.375 | |
| 72V 73W 74S | 0.270 | 1.424 | 2.044 | 1.819 | 1.477 | 1.789 | 2.662 | 0.574 | |
| 73T 72A 74F | 0.300 | 0.939 | 1.661 | 1.389 | 1.161 | 1.632 | 2.450 | 0.519 | |
| 72S 73W 74G | 0.321 | 1.012 | 1.706 | 1.375 | 0.970 | 1.653 | 2.422 | 0.411 | |
| 74S 72A 73F | 0.230 | 0.893 | 1.433 | 1.035 | 0.630 | 1.158 | 2.413 | 0.337 | |
| 74E 72A 73F | 0.259 | 0.703 | 1.558 | 1.093 | 0.575 | 1.026 | 2.395 | 0.251 | |
| 73F 72A | 0.299 | 1.051 | 1.775 | 1.171 | 0.630 | 1.118 | 2.381 | 0.399 | |
| 74T 72A 73F | 0.181 | 0.852 | 1.635 | 1.185 | 0.634 | 1.150 | 2.309 | 0.273 | |
| 55V 73F 72A | 0.328 | 0.864 | 1.566 | 1.223 | 0.661 | 1.082 | 2.264 | 0.285 | |
| 73N 72A 74F | 0.448 | 0.870 | 1.299 | 0.976 | 0.775 | 1.468 | 2.256 | 0.271 | |
| 38I 74F 72A | 0.276 | 0.787 | 1.395 | 1.162 | 0.764 | 1.343 | 2.112 | 0.503 | |
| 14L 18L 31W 73W 117L 72A | 0.201 | 1.772 | 1.640 | 1.782 | 1.085 | 0.970 | 2.059 | 0.294 | |
| 72V 73L 74W | 0.248 | 0.500 | 1.209 | 1.225 | 0.648 | 1.276 | 1.710 | 0.348 | |
| 38Y 74F 72A | 0.191 | 0.605 | 1.166 | 0.926 | 0.646 | 1.397 | 1.514 | 0.362 | |
| 74Q 72A 73F | 0.251 | 0.465 | 1.025 | 0.711 | 0.390 | 0.649 | 1.479 | 0.177 | |
| 72E 73V 74A | 0.296 | 0.478 | 0.784 | 0.735 | 0.524 | 0.736 | 1.093 | 0.234 | |
| 72H 73Y 74G | 0.205 | 0.600 | 0.591 | 0.472 | 0.140 | 0.486 | 0.986 | 0.052 | |
| 72G 73F 74N | 0.513 | 0.617 | 1.367 | 0.454 | −0.032 | 0.456 | 1.499 | −0.043 | PA & SA lowest, POA and |
| | | | | | | | | | LNA highest |
| 14L 18L 73W 117S 72A | 0.562 | 1.010 | 1.499 | 1.385 | 0.374 | 0.259 | 0.750 | −0.005 | SA ˜ zero, LNA, LA specific |
| 14Q 18S 73I 117W 72A | 0.487 | 0.878 | 0.894 | 0.784 | 0.809 | 0.559 | 0.834 | −0.044 | SA ˜ zero |
| 14L 18L 31L 73G 117V 72A | 0.440 | 1.180 | 1.139 | 1.170 | 0.031 | 0.377 | 0.180 | 0.170 | OA lowest, PUFA highest |
| 38A 106V 117A 72A | 0.418 | 1.492 | 0.541 | 0.229 | 0.368 | 0.033 | 0.121 | 0.065 | PA lowest, AA highest by 3 |
| | | | | | | | | | fold |
| 14L 18S 31L 73V 117A 72A | 0.486 | 0.951 | 0.673 | 0.534 | 0.020 | 0.028 | −0.003 | 0.007 | POA lowest |
| 38Y 117A 72A | 0.350 | 2.216 | 1.547 | 1.869 | 1.976 | 0.933 | 0.839 | 1.201 | |
| 62N 106F 117A 72A | 0.224 | 1.265 | 0.756 | 0.985 | 0.864 | 0.430 | 0.341 | 0.490 | |
| 38Y 62W 117A 72A | 0.554 | 2.353 | 1.237 | 1.848 | 1.896 | 0.316 | 0.272 | 0.854 | |
| 38Q 62I 106I 117A 72A | 0.215 | 1.656 | 0.930 | 1.288 | 1.167 | 0.550 | 0.473 | 0.663 | |

AA denotes arachidonic acid;
LNA denotes linolenic acid;
LA denotes linoleic acid;
OA denotes oleic acid;
PA denotes palmitic acid
POA denotes palmitoleic acid;
SA denotes stearic acid.

TABLE 3
Substitutions are shown with respect to SEQ ID NO: 2.

| | DR/DRAD2 | | | | | |
|---|---|---|---|---|---|---|
| Clone | Ro | AA | LNA | LA | OA | PA |
| L72A | 0.17 | 1.12 | 1.16 | 1.14 | 1.15 | 1.02 |
| L72G | 0.28 | 0.62 | 0.48 | 0.48 | 0.52 | 0.26 |
| L72M | 0.38 | 1.31 | 1.35 | 1.46 | 1.49 | 1.32 |
| A73F, L72A | 0.33 | 1.21 | 1.86 | 1.23 | 0.73 | 1.13 |
| A73I, L72A | 0.27 | 0.38 | 0.03 | −0.04 | −0.04 | −0.04 |
| D74F, L72A | 0.25 | 0.44 | 1.04 | 0.67 | 0.51 | 0.93 |
| L78F, L72A | 0.35 | 2.04 | 2.05 | 2 | 1.83 | 0.91 |
| W82F, L72A | 0.27 | 0.68 | 0.76 | 1.08 | 0.78 | 0.41 |
| R106W, | 0.24 | 1.11 | 1.03 | 1.04 | 0.99 | 0.41 |
| L72A | | | | | | |
| Y117A, | 0.28 | 1.27 | 0.62 | 1.01 | 0.93 | 0.32 |
| L72A | | | | | | |
| Y117S, | 0.36 | 1.35 | 1.07 | 1.42 | 1.19 | 0.55 |
| L72A | | | | | | |

AA denotes Arachidonic acid;
LNA denotes linolenic acid;
LA denotes linoleic acid;
OA denotes oleic acid;
PA denotes palmitic acid

TABLE 4
Substitutions are shown with respect to SEQ ID NO: 2.

| | ΔR/DRAΔ2 | | | | | | Mutations | | | |
|---|---|---|---|---|---|---|---|---|---|---|
| ID | Ro | AA | LNA | LA | OA | PA | Y14 | L38 | L72 | Y117 |
| L1P8H2 | 0.69 | −1.3 | −1.3 | −1.5 | −1.7 | −0.6 | M | M | W | wt |
| L1P7H4 | 0.62 | −1.0 | −1.1 | −1.3 | −1.4 | −0.6 | I | M | W | wt |
| L1P1C3 | 0.60 | −0.9 | −1.0 | −1.1 | −1.3 | −0.4 | M | wt | W | wt |
| L1P17A1 | 0.56 | −0.8 | −0.9 | −1.0 | −1.2 | −0.4 | I | wt | W | wt |
| L1P12E11 | 0.59 | −0.5 | −0.7 | −0.7 | −1.0 | −0.1 | L | wt | W | wt |
| L1P12F12 | 0.28 | 0.9 | 0.5 | 0.5 | 0.3 | 0.1 | L | wt | G | V |
| L1P2F7 | 0.32 | 0.9 | 0.5 | 0.6 | 0.3 | 0.1 | I | wt | G | V |
| L1P7B11 | 0.21 | 0.7 | 0.9 | 0.9 | 0.4 | 0.8 | I | I | V | wt |
| L1P12G10 | 0.35 | 0.8 | 1.0 | 0.9 | 0.5 | 0.8 | I | wt | wt | wt |
| L1P3D6 | 0.24 | 0.9 | 0.9 | 0.8 | 0.5 | 0.5 | L | wt | wt | wt |
| L1P1F7 | 0.22 | 0.9 | 1.0 | 0.9 | 0.5 | 0.5 | L | M | wt | F |
| L1P14A9 | 0.25 | 0.8 | 0.8 | 0.8 | 0.4 | 0.2 | L | wt | G | I |
| L1P11F12 | 0.27 | 1.0 | 1.2 | 1.1 | 0.7 | 0.7 | L | I | wt | wt |
| L1P17A8 | 0.24 | 1.3 | 0.7 | 0.8 | 0.7 | 0.4 | wt | M | G | F |
| L1P9G10 | 0.29 | 1.0 | 0.7 | 0.8 | 0.9 | 0.4 | wt | I | G | wt |
| L1P16G10 | 0.34 | 0.9 | 1.3 | 1.1 | 0.7 | 0.8 | W | wt | M | A |
| L1P5A10 | 0.28 | 1.5 | 0.7 | 1.2 | 1.1 | 0.4 | wt | wt | wt | A |
| L1P8D4 | 0.31 | 1.6 | 2.0 | 2.0 | 1.1 | 0.9 | M | wt | M | A |

AA denotes Arachidonic acid;
LNA denotes linolenic acid;
LA denotes linoleic acid;
OA denotes oleic acid;
PA denotes palmitic acid

TABLE 5
Substitutions are shown with respect to SEQ ID NO: 2.

| | ΔR/DRAΔ2 | | | | | | | | Sequence | | | |
|---|---|---|---|---|---|---|---|---|---|---|---|---|
| ID | Ro | AA | LNA | LA | OA | PA | POA | SA | M18 | G31 | F55 | A73 |
| L2P17H10 | 0.23 | 1.8 | 1.2 | 1.5 | 1.4 | 1.0 | 1.2 | 1.7 | G | F | WT | L |
| L2P22G6 | 0.30 | 1.8 | 2.7 | 2.6 | 1.6 | 1.9 | 2.6 | 0.9 | I | Y | WT | G |
| L2P11B4 | 0.35 | 1.8 | 2.8 | 2.6 | 1.9 | 2.2 | 3.1 | 0.7 | V | A | WT | W |
| L2P2E11 | 0.31 | 1.9 | 2.8 | 2.5 | 1.6 | 2.0 | 2.6 | 0.8 | I | F | WT | WT |
| L2P4C5 | 0.35 | 2.2 | 2.8 | 2.8 | 2.1 | 1.8 | 2.6 | 1.0 | I | Y | WT | WT |
| L2P1E6 | 0.34 | 0.4 | 0.1 | 0.1 | 0.1 | 0.0 | 0.1 | 0.2 | L | WT | WT | L |
| L2P8F11 | 0.28 | 0.4 | 0.1 | 0.0 | 0.0 | 0.0 | 0.1 | 0.3 | WT | WT | WT | I |
| L2P12G9 | 0.46 | 0.5 | 0.0 | −0.1 | 0.0 | 0.0 | 0.0 | 0.4 | L | WT | WT | V |
| L2P9E12 | 0.38 | 0.6 | 0.9 | 0.7 | 0.6 | 1.1 | 0.8 | 0.4 | L | WT | L | I |
| L2P22H10 | 0.30 | 0.9 | 1.3 | 1.1 | 0.6 | 1.1 | 1.2 | 0.4 | I | WT | G | M |
| L2P8B8 | 0.31 | 1.1 | 1.8 | 1.2 | 0.6 | 1.1 | 2.4 | 0.4 | WT | WT | WT | F |
| L2P16A3 | 0.37 | 1.0 | 1.5 | 1.1 | 0.6 | 1.1 | 1.4 | 0.3 | I | L | WT | G |
| L2P7F4 | 0.32 | 0.8 | 1.5 | 1.1 | 0.6 | 1.3 | 1.3 | 0.3 | V | V | WT | I |
| L2P21G3 | 0.36 | 1.0 | 1.4 | 1.1 | 0.6 | 1.1 | 1.4 | 0.3 | I | M | M | WT |
| L2P23G7 | 0.30 | 0.9 | 1.2 | 1.0 | 0.6 | 1.0 | 1.4 | 0.4 | V | F | L | WT |
| L2P22E6 | 0.35 | 1.0 | 1.6 | 1.3 | 0.8 | 1.3 | 1.6 | 0.4 | V | A | I | W |
| L2P18H12 | 0.36 | 1.3 | 1.9 | 1.7 | 1.0 | 1.6 | 2.1 | 0.5 | I | V | WT | G |
| L2P8A5 | 0.35 | 1.1 | 1.9 | 1.6 | 0.9 | 1.5 | 2.0 | 0.4 | V | A | W | W |
| L2P21C1 | 0.36 | 2.8 | 3.1 | 3.4 | 2.7 | 2.0 | 2.9 | 1.6 | I | Y | WT | V |
| L2P23A4 | 0.40 | 2.8 | 2.6 | 3.1 | 2.3 | 1.9 | 2.8 | 1.2 | V | I | L | V |
| L2P12C5 | 0.39 | 1.3 | 1.7 | 1.5 | 0.8 | 1.5 | 1.9 | 0.4 | I | I | M | I |
| L2P8A6 | 0.36 | 0.6 | 1.2 | 0.8 | 0.4 | 1.0 | 1.3 | 0.2 | I | V | V | V |

AA denotes Arachidonic acid;
LNA denotes linolenic acid;
LA denotes linoleic acid;
OA denotes oleic acid;
PA denotes palmitic acid;
POA denotes palmitoleic acid;
SA denotes stearic acid.

TABLE 6
Substitutions are shown with respect to SEQ ID NO: 2.

| | ΔI/Io | | | | | |
|---|---|---|---|---|---|---|
| | 1:2 UCB:BSA | | 1:1 UCB:BSA | | | |
| PROBE ID | 440 nm | 500 nm | 440 nm | 500 nm | Kd (nM) at 22° C. | Mutations |
| ADIFAB2 | −0.19 | −0.16 | −0.40 | −0.34 | — | — |
| L1P1 B4 | −0.36 | −0.35 | −0.66 | −0.64 | 590 | 14I 72W 117W |
| L1P1 C12 | −0.86 | −0.79 | −0.92 | −0.86 | 37 | 38I 72W 117W |
| L1P12 E8 | −0.45 | −0.39 | −0.79 | −0.70 | 112 | 14L 38A 72G 117F 114E |
| L1P14 D6 | −0.37 | −0.33 | −0.69 | −0.64 | 230 | 14M 72V 117W |
| L1P5 H9 | −0.34 | −0.34 | −0.58 | −0.63 | 490 | 14M 72I 117W |
| L2P22 B1 | −0.36 | −0.35 | −0.70 | −0.68 | 550 | 18Y 31V 55V 72A |
| L5P16 H4 | −0.37 | −0.31 | −0.69 | −0.64 | 390 | 126K 73F 72A |

The binding affinities of ADIFAB2 have been found to be about 10-fold greater than ADIFAB. ADIFAB2 also has an altered spectral response, making it especially useful for measurements of FFAu in blood samples (Apple et al, Clinical Proteomics, (2004) 1:41-44, U.S. patent application Ser. No. 10/670,958). The wavelengths at the maximum intensities emitted by these fluorescently-labeled I-FABP's in the absence of FFA is about 420 to 480 nm. The emission wavelengths at the maximum intensities emitted by these fluorescently-labeled I-FABP's with FFA bound are between about 495 to 580 nm. Experiments typically involve measuring the fluorescence response within both emission maxima or at wavelengths for which the effect of interfering molecules such as hemoglobin can be eliminated as described in U.S. application Ser. No. 10/670958 and PCT/US2004/030521 and the calculation of the ratio ‘R’ of the two fluorescence intensities. The baseline value for this ratio, measured in the absence of analyte, is designated R₀.

The agent may be an unbound FFA, acylglycerol, drug, drug metabolite, hormone, prostaglandin, leukotriene, sphingosine, sphingolipid, phospholipid, glycolipid, cholesterol, cholesterol derivatives, other steroids, lipid-soluble vitamin, bile salt, enzyme cofactor, retinoid such as retinoic acid and retinal, flavonoids, coumarin and coumarin derivatives, terpenoids, heme or heme metabolite, amino acid, peptide, simple or complex carbohydrate, nucleic acid or multivalent ion. Classes of unbound free fatty acids include saturated, unsaturated, monounsaturated, polyunsaturated, short chain, medium chain and long chain. Small molecules, such as small organic molecules, and other drug candidates may be obtained from combinatorial and natural product libraries. Preferably, the number of wells in the multiwell plate is between 1 and 1536. Preferably, at least some of the reagents are added to the plates using robotic liquid handling systems. Preferably, the fluorescence signal is measured from each well with a fluorescence plate reader to determine if the signals of each probe in the presence of the agent to be tested are significantly different than those in the absence of the agent to be tested.

In preferred embodiments, potentially useful molecules are selected by first selecting a chemical library to be screened. A multi-well plate is preferably prepared with an aqueous solution of an LiBP probe in most wells of the plate. Defined amounts of each molecule from the chemical library are added to wells of the plate and mixed with probe in the wells. For comparison the molecules from the chemical library are also added to wells with aqueous buffer, but without probe. The fluorescence of each well is determined using a fluorescence plate reader. The plate is screened for molecules which change the fluorescence of the probe relative to probe without a molecule from the library. The fluorescence of the library molecules without probe is also measured to determine if there is any interfering fluorescence. Any interfering fluorescence can be used to correct the probe response. Molecules which significantly change the fluorescence of the probe are selected for further testing.

Some embodiments of the invention are directed to a method for screening molecular libraries for compounds which bind to fluorescently labeled LiBPs including fluorescently labeled FABPs such as ADIFAB, ADIFAB2, and other fluorescently labeled FABPs as shown in Tables 1-6. In preferred embodiments, the probes are labeled with acrylodan, preferably while bound to a solid support as described in U.S. application Ser. No. 11/085,792, which is incorporated herein by reference. However other fluorescent labels may also be used such as but not limited to danzyl aziridine, 4-[N-[(2-iodoacetoxy)ethyl]-N-methylamino]-7-nitrobenz-2-oxa-1,3-diazole ester (IANBDE), and 4-[N-[(2-iodoacetoxy)ethyl]-N-methylamino-7-nitrobenz-2-oxa-1,3-diazole (IANBDA). Any fluorescent label may be used in the practice of the invention as long as a measurable difference may be detected upon binding of a free fatty acid or other analytes.

Probes useful in embodiments of the invention are shown in Tables 1-6. The indicated substitutions are with reference to SEQ ID NO: 2. The measurement of whether or not the agent is capable of binding to the probe, as determined by a change in fluorescence, is essentially a prescreen to select for potentially useful lead compounds. These measurements are performed in high throughput formats, using a fluorescent plate reader and multi-well (1 to 1536) plates. The fluorescently labeled FABPs (probes) respond to binding of FFA and other ligands by a change in the ratio of emission fluorescence at 2 wavelengths. Using these probes, molecular libraries which may include potential drug candidates, are screened for molecules that bind to the probe with high affinity. By this means non-FFA (or non-natural ligand) molecules that bind to probes with high affinity are discovered. These molecules are potentially useful lead compounds for drug development and/or further screening as described below.

The intensity ratio (“R” value) is determined as follows. The ratio is calculated using the following formula:
R=F_{λ1}/F_{λ2}
wherein, F_{λ1} is the measured fluorescence intensities (intensity of sample with probe present minus intensity of sample without probe present) at wavelength 1 and F_{λ2} is the measured fluorescence intensities (intensity of sample with probe present minus intensity of sample without probe present) at wavelength 2. Then, for a given probe, ΔR, the difference in R value between the measurement in the presence of the test compound (agent) and in the absence of the test compound (agent), is calculated as follows:
ΔR=R_{+agent}−R₀
In some embodiments, the ΔR value may then be compared to some reference, for example, an agent with a known affinity for a given probe, by ΔR/ΔR_{reference}. Measurements of fluorescence intensities are obtained using standard techniques.

Preferably, the fluorescence intensities at two or more wavelengths are measured in each well and the intensity ratios at all combinations of the two or more wavelengths are evaluated to determine if the ratios for each agent are significantly different than those of a reference ligand or agent. By this method, agents may be identified that have different specificities in their fluorescence response to different probes as compared to a reference agent. In preferred embodiments, the reference agent may be the natural ligand such as an unbound free fatty acid. Other methods for comparing changes in fluorescence with and without agent can also be used.

In preferred embodiments, the high affinity non-natural ligand molecules that bind to a probe, as discussed above, are then screened for binding to wild type LiBPs (LiBP that are not covalently labeled with a fluorescent molecule) to reveal which molecules bind to the wild type LiBPs with high affinity. This screen may occur in two phases:

Phase 1 is a qualitative phase in which the candidate inhibitors are tested for their ability to displace a fluorescent molecule that binds non-covalently to the wild type LiBP and which reveals measurable fluorescence when bound to the LiBP and a different fluorescence when not bound (in the aqueous phase). The fluorescent molecule is any fluorescent molecule that can bind to a wild type LiBP. Preferably, the fluorescent molecule is a molecule that binds non-covalently in the binding site of the LiBP. In the case of a FABP, the fluorescent molecule may advantageously be a molecule that binds in the binding pocket of the FABP for FFA. In some preferred embodiments, the fluorescent molecule is a fluorescently labeled FFA.

Phase 2 is a quantitative phase when the values of the affinities for the successful candidates from Phase 1 for binding to the LiBP are determined. In preferred embodiments, binding affinities are determined by titrating a mixture of LiBP and probe with a candidate molecule, using the probe to determine the amount bound to LiBP. This method uses the same principle as described for determination of FABP affinities for FFA using ADIFAB to monitor binding as discussed in Richieri GV et al (1999) Mol Cell Biochem 192: 87-94 which is incorporated herein by reference. To calibrate a probe for a given agent, a known amount of the agent is titrated into a cuvette with the probe, measuring the R value, determined as described above, after each addition. Once the amount of agent binding to the cuvette walls has been subtracted out (see below) one can fit the titration curves to determine the parameters (Kd, Q, and Rmax) [as described in Richieri GV et al (1992) J. Biol. Chem 267:23495-23501 and Richieri G V et al (1999) Mol Cell Biochem 192: 87-94] that govern the response of the probe to the agent molecule. K_{d} is determined by fitting results to titration curves with K_{d}'s generated using the equation below.

$$[ agent ] = \frac{Kd \times Q \times ( R - {R}_{0} )}{( R max - R )}$$

where Q=I_{f}/I_{b};
I_{f} and I_{b} are the probe intensities at 432 (for ADIFAB) with zero (free) and saturating (bound) concentrations of agent, respectively;
R is the measured ratio of F_{λ1}/F_{λ2} (505:432 nm for ADIFAB) with blank intensities subtracted;
R₀ is the measured ratio of F_{λ1}/F_{λ2} (505:432 nm for ADIFAB) with no agent present; and
Rₘₐₓ is the measured ratio of F_{λ1}/F_{λ2} (505:432 nm for ADIFAB) when the probe is saturated.

The amount of agent binding to cuvette walls may be determined as follows. To determine the amount of wall binding, the change in R value is measured upon transferring a sample containing agent from one cuvette to another. First the R0 value is determined in a sample containing the probe in buffer without the agent. A small amount of the agent is added. After waiting 1-10 min, R is measured. The contents are transferred to a second cuvette. After equilibrium is reached R′ is measured. From the difference between R and R′, the fraction bound (BF) to the walls can be determined as: BF=(R−R′)/(R−R0). The amount bound differs for different agents, buffers, temperatures, and cuvette materials.

For example, ADIFAB can be used to determine the binding of an agent to an unlabeled LiBP. In the case of a single class of binding sites, the titration data can be analyzed by the Scatchard method as:

$$\frac{{[ agent ]}_{bound}}{\frac{{[ LiBP ]}_{total}}{{[ agent ]}_{unbound}}} = \frac{{[ agent ]}_{bound}}{{{K}_{d}^{′} [ LiBP ]}_{total}} + \frac{n}{{K}_{d}^{′}}$$

where n is the number of agent binding sites per LiBP monomer, and K_{d}′ is the binding affinity of the agent to the LiBP. Plotting the data as bound agent/LiBPₜₒₜₐₗ vs. bound agent/LiBPₜₒₜₐₗ/unbound agent yields a straight line with the slope of the line equal to −1/K_{d}′, and the x axis intercept equal to n. For multiple binding sites of different affinities such a plot is non-linear.
A: amount of agent added
B: measured R value
C: unbound agent
D: amount of agent binding to ADIFAB
E: amount of agent bound to the LiBP being tested
F: [agent]_{bound} (abscissa for a Scatchard plot)
   [LiBP]ₜₒₜₐₗ
G: [agent]_{bound} (ordinate for a Scatchard plot)
   [LiBP]ₜₒₜₐₗ/[agent]_{unbound}

Unbound agent in C is determined by the following formula and R values from column B:

$$[ unbound agent ] = {K}_{d} \times 19.5 \times \frac{( R - {R}_{0} )}{( 11.5 - R )}$$

Agent binding to ADIFAB (D) is determined using R values from B and the following equation:

$${[ ADIFAB ]}_{bound} = \frac{{( ADIFAB ]}_{total} \times 19.5 \times ( R - {R}_{0} )}{11.5 - R + 19.5 \times ( R - {R}_{0} )}$$

Agent bound to the LiBP (E) is determined by A-C-D.

The qualitative phase and the quantitative phase described above are independent of each other and may be carried out separately. In some embodiments, when an appropriate fluorescent molecule that binds to the wild type LiBP is not available, screening may be carried out on the basis of quantitative phase 2. In preferred embodiments, candidate molecules are first screened in the qualitative screen of phase 1. Successful candidate molecules from phase 1 are then screened in the second quantitative phase.

The successful candidate of 2) can be tested to determine its ability to permeate cell membranes and thereby gain access to the cell cytoplasm where the intracellular LiBPs are located. Candidate drugs discovered by the above method can be tested for their effects on various aspects of trafficking and metabolism of appropriate lipid metabolites by methods familiar to one skilled in the art.

In a preferred embodiment molecular libraries are screened for binding to a probe derived from a FABP. In a preferred embodiment, the molecular library is screened using a probe such as those described in Tables 1-6 and also in U.S. application Ser. No. 11/085,792, filed Mar. 21, 2005, incorporated by reference. Yet more preferably, the probe is ADIFAB or ADIFAB2 as described above. Unbound and ADIFAB-bound FFA concentrations are determined from the ratio of emitted fluorescence at 505 to 432 nm upon excitation at 386 nm.

In a preferred embodiment the qualitative phase (1) of testing is carried out using a multi-well plate prepared with a wild type LiBP and a fluorescent probe in the wells. This plate is screened with the (agent) molecules which were pre-selected for further testing. The probe fluorescence of each well is measured with a fluorescence plate reader to determine the degree to which molecules bind to the wild type LiBP as indicated by a change in the fluorescence of the probe. A binding constant for the LiBP is determined by titrating the wild type LiBP and probe with the selected molecules. High affinity molecules are then identified from the binding constants determined by the methods as described above.

In some preferred embodiments, selected agents are tested for their ability to permeate cells of interest. Briefly, the probe may be microinjected or electroporated into cells of interest. Permeation of the agent molecule is determined from the change in the fluorescence of the intracellular probe after addition of the agent molecule to the extracellular medium for monitoring FFA permeation of cells. In preferred embodiments, cells of interest include prokaryotic and eukaryotic cells. Eukaryotic cells may include plant cells, insect cells or mammalian cells. More preferably, the cells of interest are human cells. Permeant molecules are then further screened to determine their effects on cellular metabolism. For example, for FFA, blocking a FABP from binding FFA might reduce rates of lipolysis, esterification or ATP production.

### EXAMPLE 1

Five identical 96-well plates were prepared with 0.5 μM ADIFAB and 1% dimethyl sulfoxide in 200 μL aqueous buffer (20 mM HEPES, 140 mM NaCl, 5 mM KCl, 1 mM Na₂HPO₄, pH 7.4) to test assay reproducibility, precision, and quality. Wells were excited at 386 nm and the emission detected at 432 and 505 nm with a fluorescence plate reader connected via fiber optic cables to a standard Spex spectrofluorometer. The emission ratio (505/432), which determines the amount of bound ADIFAB, was calculated for each well. A positive control (4 μM sodium oleate (OA)) was added to each well and the ratio was measured again. The average ratio (ρ), standard deviation (σ), coefficient of variation (CV), signal-to-background (S/B) and Z′-factor were calculated for each plate (Table 7). The Z′-factor, as defined below, provides a quantitative assessment of the assay [Zhang, J., et al J. Biomol. Screen. 4, 67-73 (1999)].

$${Z}^{′} = 1 - \frac{3 {σ}_{C +} + 3 {σ}_{C -}}{\vert {ρ}_{C +} - {ρ}_{C -} \vert}$$

Subscripts C+ and C− refer to positive (with OA) and negative (no OA) controls, respectively. An ideal assay has a Z′-factor of 1; assays with values between 0.5 and 1 are considered ecellent. The S/B values were calculated as ρ_{C+}/ρ_{C-}.

TABLE 71
Plate statistics for ADIFAB screening assay with 4 μM OA.

| | Negative | | | Positive | | | | |
|---|---|---|---|---|---|---|---|---|
| | Control (−OA) | | | Control (+OA) | | | | |
| Plate | ρ_{c-} | σ_{c-} | CV % | ρ_{c+} | σ_{c+} | CV % | S/B | Z′ |
| 1 | 0.215 | 0.006 | 2.8 | 1.81 | 0.09 | 5.2 | 8.42 | 0.819 |
| 2 | 0.241 | 0.013 | 5.6 | 1.68 | 0.06 | 3.7 | 6.97 | 0.848 |
| 3 | 0.232 | 0.008 | 3.5 | 1.83 | 0.12 | 6.7 | 7.89 | 0.760 |
| 4 | 0.222 | 0.009 | 3.9 | 1.77 | 0.07 | 3.8 | 7.97 | 0.847 |
| 5 | 0.228 | 0.009 | 3.8 | 1.84 | 0.15 | 8.0 | 8.07 | 0.704 |

### EXAMPLE 2

A small molecule library was screened for binding to ADIFAB in 96-well plates with a fluorescence plate reader connected via fiber optic cables to a standard Spex spectrofluorometer. Each well contained 200 lL aqueous buffer (20 mM HEPES, 140 mM NacC, 5 mM KCl, 1 mM Na₂HPO₄, pH 7.4), 0.5 μM ADIFAB, and 5 μM screening compound. Wells were excited at 386 nm and the emission detected at 432 and 505 nm. The emission ratio (505/432), which determines the amount of bound ADIFAB, was calculated for each well before and after the addition of screening compound.

Preliminary hits were detected by comparison of the change in the ratio (ΔR) after addition of the screening compound with that for 4 μM oleate (ΔR=1.8). Table 8 shows the results for an example plate of 80 screening compounds. The largest response from this plate was for well A9 and was significantly greater than the positive control. The affinity of this compound for ADIFAB was confirmed and quantitatively determined (K_{d}=90 nM) by fluorescence titration. ADIFAB was then used to determine the dissociation constants for binding of this compound to rat intestinal (K_{d}=12 nM) and mouse adipocyte (K_{d}=11 nM) FABP.

TABLE 8
Example screening results for binding to ADIFAB.

| Well | |
|---|---|
| Position | ΔR |
| A2 | 0.011 |
| A3 | 0.032 |
| A4 | 0.012 |
| A5 | 0.014 |
| A6 | 0.020 |
| A7 | 0.047 |
| A8 | 0.016 |
| A9 | 2.665 |
| A10 | 0.000 |
| A11 | 0.003 |
| B2 | 0.024 |
| B3 | 0.023 |
| B4 | 0.021 |
| B5 | 0.022 |
| B6 | 0.012 |
| B7 | 0.014 |
| B8 | −0.009 |
| B9 | −0.008 |
| B10 | 0.465 |
| C2 | 0.020 |
| C3 | 0.010 |
| C4 | 0.033 |
| C5 | 0.011 |
| C6 | 0.014 |
| C7 | 0.010 |
| C8 | 0.013 |
| C9 | −0.007 |
| C10 | 0.001 |
| C11 | −0.017 |
| D2 | 0.010 |
| D3 | 0.026 |
| D4 | −0.002 |
| D5 | 0.010 |
| D6 | 0.004 |
| D7 | −0.009 |
| D8 | −0.013 |
| D9 | −0.002 |
| D10 | 0.013 |
| E2 | 0.021 |
| E3 | 0.008 |
| E4 | 0.001 |
| E5 | −0.013 |
| E6 | 0.005 |
| E7 | −0.006 |
| E8 | 0.013 |
| E9 | 0.014 |
| E10 | 0.017 |
| E11 | 0.001 |
| F2 | 0.022 |
| F3 | 0.018 |
| F4 | 0.006 |
| F5 | 0.012 |
| F6 | 0.017 |
| F7 | −0.013 |
| F8 | −0.008 |
| F9 | 0.210 |
| F10 | 0.006 |
| G2 | 0.017 |
| G3 | 0.010 |
| G4 | 0.012 |
| G5 | 0.002 |
| G6 | 0.024 |
| G7 | 0.044 |
| G8 | 0.004 |
| G9 | 0.012 |
| G10 | −0.130 |
| G11 | 0.011 |
| H2 | 0.029 |
| H3 | 0.032 |
| H4 | 0.017 |
| H5 | 0.032 |
| H6 | 0.301 |
| H7 | 0.015 |
| H8 | 0.020 |
| H9 | 0.006 |
| H10 | 0.007 |

### EXAMPLE 3

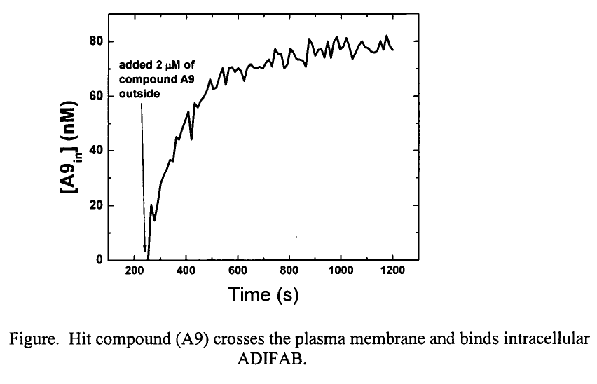

The plasma membrane permeability of the hit compound (A9) from Example 2 was determined using 3T3-F442A preadipocyte cells loaded with ADIFAB. The syringe-loading technique [Clarke, M. S. F., McNeil, P. L., J. Cell Sci. 102, 533-541 (1992)] was used to introduce ADIFAB into the cytosol of the preadipocytes. A suspension of 105 loaded cells in 1.5 mL aqueous buffer (20 mM HEPES, 140 mM NaCl, 5 mM KCl, 1 mM Na2HPO4, pH 7.4) was prepared in a continuously mixing glass cuvette. The ADIFAB fluorescence emission at 432 and 505 nm was recorded every 12 seconds while exciting at 386 nm using a standard Spex spectrofluorometer. After 252 seconds, 2 tM of compound A9 was added to the extracellular milieu. The ADIFAB emission ratio (505/432) increased after the addition of the hit compound indicating that the compound can permeate the plasma membrane and bind to intracellular ADIFAB. The intracellular concentration of compound A9 was calculated from the ADIFAB ratio, and the influx time course is shown in FIG. 1. Membrane permeability is a requisite property of any potential therapeutic that inhibits intracellular lipid binding proteins. A control permeability experiment with dimethyl sulfoxide showed no increase in ADIFAB fluorescence.

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A method of identifying agents with high affinity for an intracellular lipid binding protein (LiBP) probe, comprising the steps of:
(a) measuring a first fluorescence of a fluorescently labeled LiBP probe;
(by incubating the LiBP probe with the agent;
(c) measuring a second fluorescence;
(d) comparing the first fluorescence of the LiBP probe in the absence of the agent to a second fluorescence in the presence of the agent;
(e) selecting agents which affect a difference between the first fluorescence and the second fluorescence, and
(f) identifying agents which have affinity for the LiBP probe,
wherein a magnitude of a difference between the first fluorescence and the second fluorescence is indicative of the affinity of the agent for the LiBP probe.

2. The method of claim 1, wherein the LiBP probe is ADIFAB or ADIFAB2.

3. The method of claim 1, wherein the LiBP probe is a variant of the amino acid sequence shown as SEQ ID NO: 2 comprising one or more substitutions, insertions and/or deletions in the amino acid sequence shown as SEQ ID NO: 2.

4. The method of claim 3, wherein the LiBP probe is selected from the group consisting of any one of the probes shown in Tables 1-6.

5. The method of claim 1, wherein the agent is a drug candidate.

6. A method of screening for an agent that modulates the binding function of an intracellular lipid binding protein (LiBP), comprising the steps of:
(a) reacting a wild type LiBP with a fluorescence indicator, wherein the fluorescent indicator is non-covalently bound in a binding pocket of the wild type LiBP to form a LiBP binding complex;
(b) contacting an agent to be tested with the LiBP complex; and
(c) identifying whether the agent displaces the fluorescence indicator, thereby changing fluorescence.

7. The method of claim 6, further comprising:
(d) titrating the wild type LiBP with the fluorescent indicator to determine the binding constant of the fluorescent indicator;
(e) titrating the wild type LiBP with the agent selected in step (c) to determine a binding constant for each selected agent by using a competition assay and the binding constant of the fluorescent indicator; and
(f) evaluating the binding constants to identify agents that modulate the binding function of the LiBP.

8. The method of claim 6, wherein the LiBP is a fatty acid binding protein.

9. The method of claim 1, further comprising:
(g) providing a LiBP complex comprising a wild type LiBP and a fluorescent indicator non-covalently bound in a binding pocket of the wild type LiBP;
(h) contacting the identified agent of step (f) with the LiBP complex of step (g); and
(i) identifying whether the agent displaces the fluorescence indicator, thereby changing fluorescence.

10. The method of claim 6, wherein the fluorescence indicator comprises a fatty acid labeled with a fluorescent indicator.

11. The method of claim 7, wherein the wild type LiBP is a fatty acid binding protein.

12. A method of screening for an agent comprising:
adding a composition comprising a wild type LiBP and a probe to at least some wells of a multi-well plate;
adding test agents to the wells;
measuring fluorescence of each well to determine the degree of binding of each agent to the wild type LiBP;
selecting for agents that bind to the wild type LiBP;
titrating the wild type LiBP and the probe with the selected agents to determine binding constants; and
identifying high affinity agents.

13. The method of claim 12, wherein the probe comprises an LiBP covalently labeled with a fluorescent molecule.

14. The method of claim 13, wherein the LiBP of the probe is the same as the wild type LiBP.

15. The method of claim 13, wherein the LiBP probe is ADIFAB or ADIFAB2.

16. The method of claim 13, wherein the LiBP probe is a variant of the amino acid sequence shown as SEQ ID NO: 2 comprising one or more substitutions, insertions and/or deletions in the amino acid sequence shown as SEQ ID NO: 2.

17. The method of claim 16, wherein the LiBP probe is selected from the group consisting of any one of the probes shown in Tables 1-6.

18. A method of selecting for high affinity agents which are permeant to cells of interest comprising:
transfecting a probe into a cell;
determining the ability of an identified or selected agent enter the cell by monitoring the change in probe fluorescence after adding the agent to the outside of the cell; and
selecting for high affinity agents which are permeant to cells of interest.

19. The method of claim 18, wherein the cell is a mammalian cell.

20. The method of claim 18, wherein transfection is by microinjection, electroporation, use of lipid or peptide transfection reagents, or mechanical membrane disruption as in scrape, scratch, bead, or syringe loading.

21. The method of claim 18, wherein the probe is a variant of the amino acid sequence shown as SEQ ID NO: 2 comprising one or more substitutions, insertions and/or deletions in the amino acid sequence shown as SEQ ID NO: 2.

22. A method of selecting for high affinity agents which are permeant to cells of interest comprising:
transfecting a probe into a cell;
determining the ability of an identified or selected agent to enter the cell by monitoring the change in probe fluorescence after adding an agent identified by the method of any one of claims 1, 4, 5, or 9 to the outside of the cell; and
selecting for high affinity agents which are permeant to cells of interest.
